(19) United States
(12) Patent Application Publication
(10) Pub. No.: US 2006/0257850 A1
(43) Pub. Date: Nov. 16, 2006

(54) Methods of treatment and diagnosis of patients with hepatitis c infection

(76) Inventors: Adrian Hill, Oxford (GB); Mark Thursz, London (GB); Susanne Knapp, London (GB)

Correspondence Address: NIKOLAI & MERSEREAU, P.A., 900 SECOND AVENUE SOUTH, SUITE 820, MINNEAPOLIS, MN, 55402, US

(21) Appl. No.: 10/511,415

(22) PCT Filed: Apr. 15, 2003
(86) PCT No.: PCT/GB03/01625

§ 371 Date: Jul. 1, 2005

(30) Foreign Application Priority Data
Apr. 19, 2002 (GB) 0208928.2

(51) Int. Cl. C12Q 1/70; C12Q 1/68; A61K 39/29

(52) U.S. Cl. 435/5; 435/6; 514/12; 424/189.1

(57) ABSTRACT

Use of a compound capable of modulating the level of activity of the OAS gene and/or activity of the OAS protein, in the manufacture of a medicament for the treatment of a patient with or at risk of hepatitis C infection, wherein the compound is not an interferon or an isoprenoid, such as geranylgeranylacetone (GGA). A method of screening for compounds for treating HCV infection, wherein a cell is treated with a test compound and any change in OAS gene activity and/or OAS protein activity or level is assessed, wherein the compound is not an interferon or an isoprenoid, such as geranylgeranylacetone (GGA). Use of a compound capable of modulating the level of activity of the RNAse L gene and/or activity of the RNAse L protein, in the manufacture of a medicament for the treatment of a patient with or at risk of hepatitis C infection, wherein the compound is not an interferon or an isoprenoid, such as geranylgeranylacetone (GGA). A method of screening for compounds for treating HCV infection, wherein a cell is treated with a test compound and any change in RNAse L gene activity and/or RNAse L protein activity or level is assessed, wherein the compound is not an interferon or an isoprenoid, such as geranylgeranylacetone (GGA).

The present invention relates to the treatment and diagnosis of patients with hepatitis C infection.

The hepatitis C virus is a single stranded RNA virus, encoded by a genome of approximately 9,500 nucleotides. It is a member of the Flaviviridae family, being most closely related to GBV-A, GBV-B and GBV-C, the other Hepaciviruses (Sharara et al (1996) Ann. Intern. Med.; 125(8):658-668). The virion, which has been visualized with electron microscopy in lymphoblastoid cell cultures (Callan et al (1998) J. Exp. Med.; 187:1395-1402), consists of the positive strand RNA molecule, surrounded by the core (nucleocapsid) and two envelope proteins (E1,E2). In common with all members of the Flaviviradae, the HCV genome contains a long translational open reading frame (ORF), which encodes a single polypeptide that is cleaved co- and post-translationally by cellular and viral proteinases to yield a number of structural and non-structural proteins (Gallimore et al (1998) J. Exp. Med.; 187:1383-1393). The structural proteins comprise of core, E1 and E2. The non-structural proteins, which are not expected to be constituents of the virion, are NS2, NS3, NS4a, NS4b, NS5a, and NS5b. These have a variety of functions, necessary for viral replication, including proteinase (NS2, NS3), NTPase/helicase (NS3) and RNA-dependent RNA polymerase (RdRp) (NS5b) activities. Translation of the HCV ORF is directed via a 340 nucleotide long 5′ non-coding region (5′NCR), functioning as an internal ribosome entry site (IRES). There is a second non-coding region at the 3′ end of the ORF (3′NCR) which includes a poly(U) tract.

The immune response to non-cytopathic viral infections can lead to three outcomes: clearance of the virus; long term control or suppression of the virus; or uncontrolled viraemia with the possibility of progressive disease. The majority of patients infected with hepatitis C virus or human immunodeficiency virus fall into this latter category. Hepatitis C virus (HCV) represents a more recently recognised global health problem with 4-6% of certain populations chronically infected (Choo et al (1989) Science; 244; 359-362). It is estimated that in excess of 170 million individuals worldwide are persistently infected with this virus. Between 20 and 30% of these individuals will develop hepatic cirrhosis and its long-term sequelae such as hepatocellular carcinoma (Tong et al (1995) N. Eng. J. Med.; 332; 1463-1466). HCV is now the most common indication for liver transplantation in Europe and North America (Bismuth et al (1992) Clin. Transpl.; 1992; 161-166). However, not all individuals who are exposed to the virus develop a persistent infection (Alter et al (1997) Hepatology; 26; 29S-33S; Crowe et al (1995) Gastroenterology; 108; A1054; Kenny (1999) N. Eng. J. Med.; 340; 1228-1233). In a number of reports both biochemical and virological recovery from infection has been clearly documented. Although there is some variability in the reported frequency, self-limiting infection occurs in 20 to 25% of individuals.

At present treatment of HCV infection is limited to interferon-monotherapy or a combined interferon-ribavirin treatment, or pegylated interferon in combination with Ribavirin. Among those patients with persistent infection and subjected to treatment with interferon-monotherapy, sustained response (SR) is achieved in 5-30% of treated patients (Hoofnagle et al, (1986) N. Engl. J. Med.; 315; 1575-1578). Pegylated interferon and Ribavirin combination therapy may achieve a sustained response rate of 54% overall, 80% in patients with viral genotypes 2 or 3 but only 42% in patients infected with HCV viral genotype 1 (Manns M P et al Lancet 2001 Sep. 22;358(9286):958-65)

Interferons are a family of related cytokines that mediate a range of diverse functions including antiviral, antiproliferative, antitumour, and immunomodulatory activities. The pleiotropic activities of interferons are mediated primarily through the transcriptional regulation of many downstream effector genes, termed Interferon Stimulated Genes (ISGs) (Der et al (1998) Proc. Natl. Acad. Sci. USA; 95; 15623-15628). Among these are genes that are responsible for creating an antiviral state in the cell, which involves the induction of enzymes that actively degrade viral nucleic acid and inhibit viral protein synthesis (Enomoto et al (1995) J. Clin. Invest.; 96; 224-230). There are several potentially key genes in this process, among those being RNA-dependant protein kinase (PKR), 2′-5′-oligoadenylate-synthetase (OAS) and myxovirus (influenza) resistance 1 (MxA).

Amongst these interferon-induced genes, 2′,5′-oligoadenylate synthetase (OAS) may play an important role in a potent viral defence mechanism that can act against specific species of RNA viruses. Upon binding to dsRNA, OAS catalyses the formation of 2′,5′ linked oligoadenylate (2-5A) and activates RNAse L, a latent endoribonuclease, which becomes activated by binding 2-5A.

The expression, regulation, and function of the OAS and 2-5A-dependent RNAse L has been characterised (Silverman R H, Cayley P J, Knight M, Gilbert C S, Kerr I M. Eur J Biochem 1982 May; 124(1):131-8.; Gribaudo G, Lembo D, Cavallo G, Landolfo S, Lengyel P. J Virol 1991 April; 65(4): 1748-57; Sen G C, Lebleu B, Brown G E, Kawakita M, Slattery E, Lengyel P. Nature 1976 Nov. 25; 264(5584): 370-3; Yan C, Sehgal P B, Tamm I. Proc Natl Acad Sci USA 1989; 86(7):2243-7. Three size forms of the human OAS enzyme family have been identified: OAS1, OAS2 and OAS3, the genes of which are clustered over ˜130 kb on chromosome 12. The 5′-regions of OAS1, OAS2 and OAS3 genes each contain DNA sequences which interferon-mediated transcriptional complexes bind to, to promote gene transcription. Each family member shares a conserved protein domain within the N-terminal region of the protein, OAS1 having one such domain, OAS2 having two domains, and OAS3 having three copies of this domain. Each of the family members accumulate in different cellular locations, require different amounts of dsRNA to be activated, and catalyse the formation of differently sized 2-5A products. The significance of this has yet to be assessed.

As discussed above, OAS proteins generate 2-5A molecules which in turn activate RNAse L, a latent endoribonuclease. 2-5A molecules are considered to be required for the conversion of RNAse L from an inactive monomeric form to an active dimeric form. In the active state RNAse L degrades both viral and cellular RNA. RNAse L is constitutively expressed in most cell types; however, gene expression can be enhanced through the application of interferons. The importance of RNAse L in the viral resistance system of mammalian cells is emphasised by studies of mice with defective RNAse L genes. In this case mutant mice were more susceptible to viral infections, with an added factor of displaying developmental abnormalities associated with a disrupted apoptotic system (Zhou et al (1997) EMBO; 16; 6355-6363).

There are now assay systems available for measuring RNAse L activity both from intact human cells and of recombinantly purified protein (Rusch et al (2001) Methods Enzymol.; 342; 10-20). In addition recent studies have shown a human OAS gene can be expressed and purified in vitro leading to a better understanding of the mode of action of this protein (Sarkar and Sen (1998) Methods; 15; 233-242).

Although over expression of OAS1 cDNA in human cells leads to a greater antiviral state, the viral response is selective for specific classes of dsRNA viral families. For example, over-expression of OAS1 cDNA leads to resistance to encephalomyocarditis virus replication, but not herpesvirus replication. Hence elevating the level of OAS proteins in a cell can lead to greater resistance of a cell to viral replication, but there is a great degree of variation in the response of the cell to different viruses.

With HCV infection and treatment, OAS levels seem to be unconnected to the response of the patient to interferon-α treatment. Murashima et al (2000) J. Med. Virol.; 62; 185-190 concluded that OAS activity does not seem related to long-term virological response to interferon-α therapy. Similarly Donohue et al (1993) Br. J. Haematol.; 83; 491-497 reported that OAS mRNA levels increased after interferon-α therapy, but that OAS levels did not correlate to the response of the patient to the treatment. Solina et al (1993) Liver; 13; 253-258 found that HCV infection by itself induces elevated OAS levels. They also reported that OAS levels showed the most elevated and stable increase in patients who did not respond to interferon-α treatment and, hence, monitoring of OAS activity in the serum does not predict the response to interferon. Finally, Yu et al (2000) Hepatology; 32; 1089-1095 found that OAS levels in HCV infected liver samples were not significantly different to levels found in normal liver sample, which contradicts the earlier findings of Solina et al.

Thus, OAS appears to have no function in mediating the action of interferon-α during HCV therapy and, overall, the role of OAS in the response of a patient to HCV is very unclear. In contrast, the interferon-α induced RNA-dependant protein kinase (PKR) and myxovirus (influenza) resistance 1 (MxA) genes are considered to play a role in the elimination of HCV in patients treated with interferon-α (Chieux et al (1998) J. Virol. Methods; 70; 183-191; Yu et al (2000) Hepatology; 32; 1089-1095).

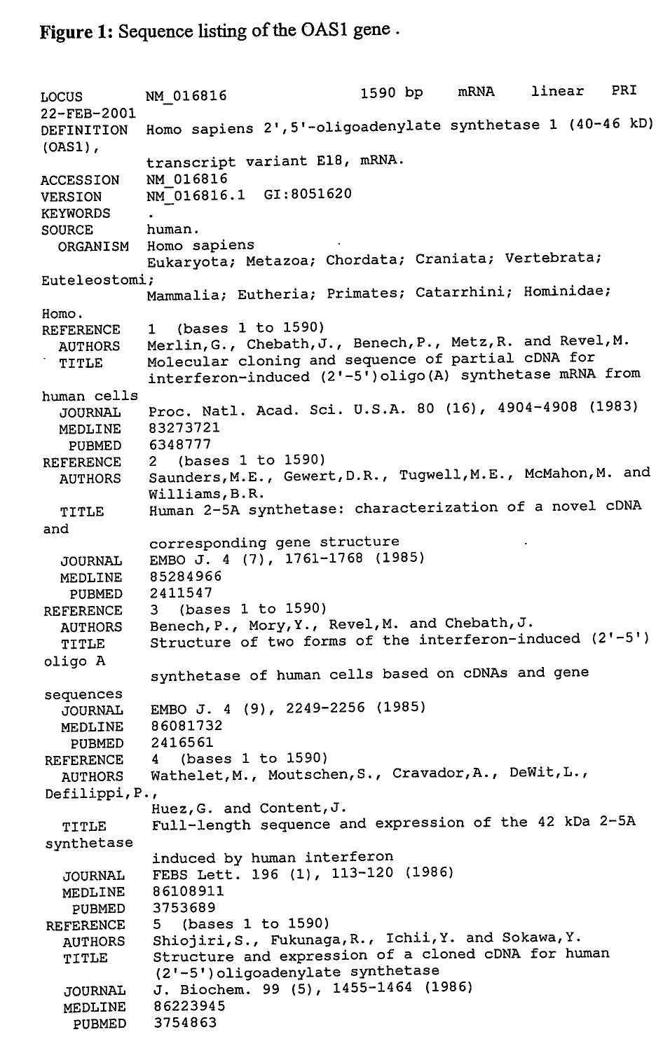

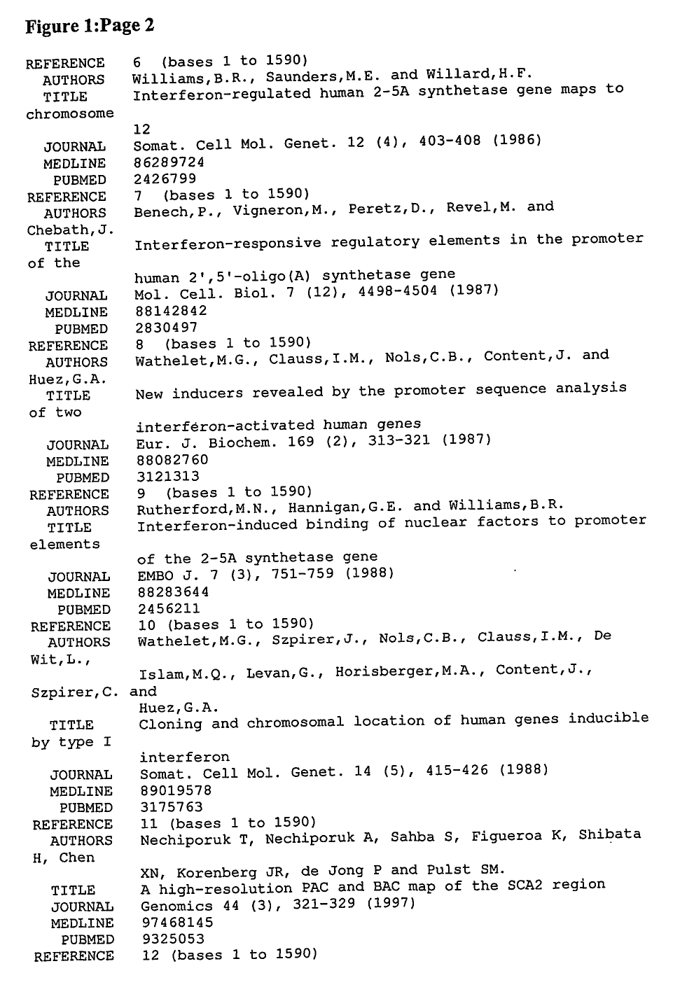

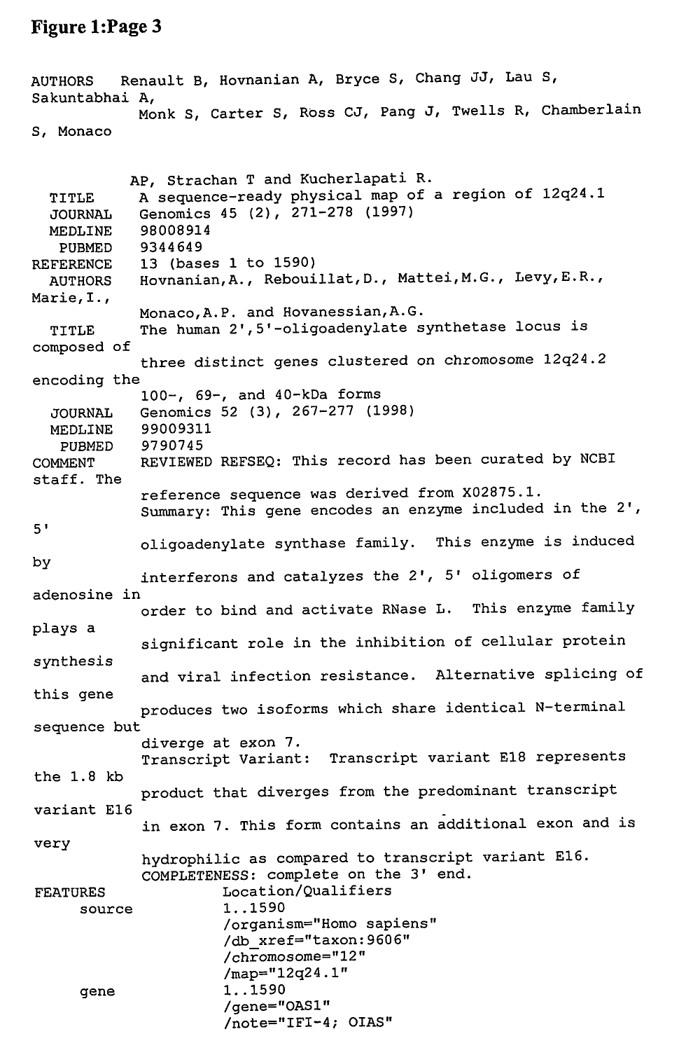

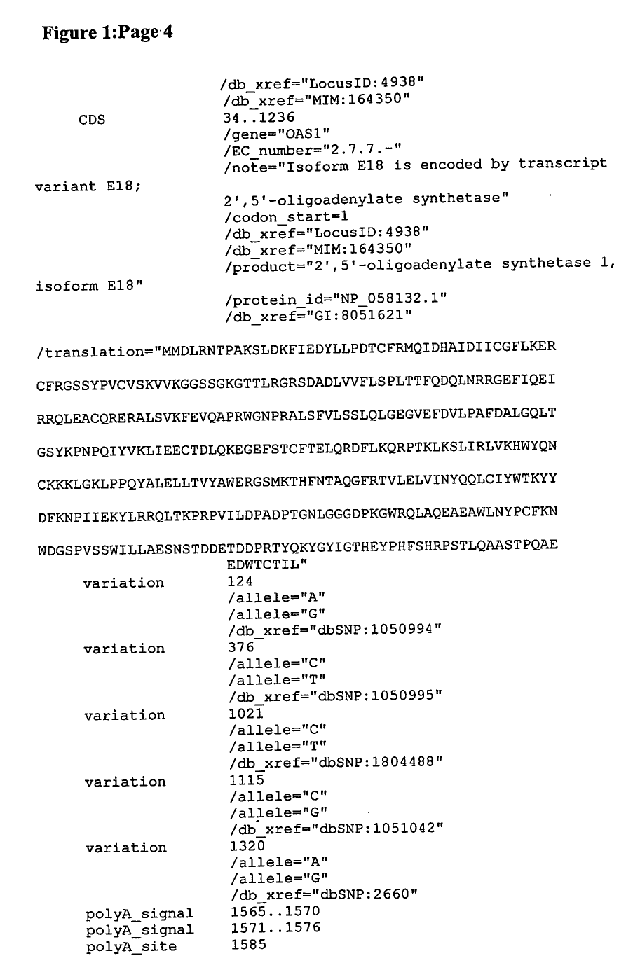

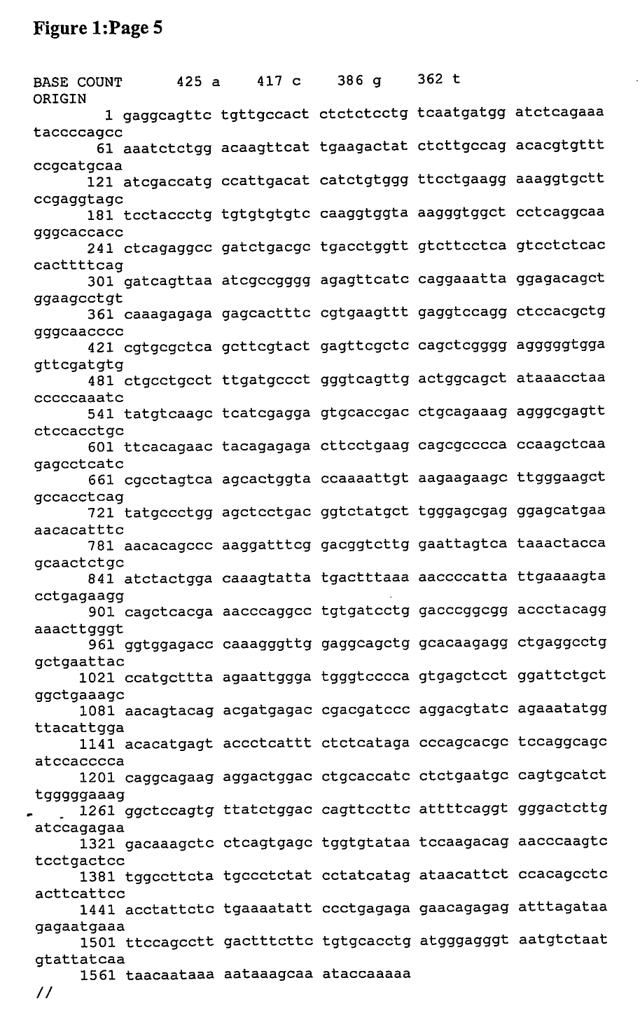

We have surprisingly found that OAS genotype is linked with the outcome of HCV infection. We have identified a polymorphism in the OAS gene which is associated with self-limited infection of HCV. Patients who have a GG genotype at position 84 in the untranslated 3′end of exon 8 of OAS-1 (position 347 of Genbank accession number Ml 1810) are more likely to have persistent HCV infection in comparison to those with an AG or AA genotype at the same position. This corresponds to position 1320 of the sequence of OAS1 given in FIG. 1. We provide methods of treatment and diagnosis of patients with or at risk of HCV infection and compounds and compositions that may be useful in such methods.

A first aspect of the invention provides the use of a compound and/or composition that has the property of modulating the level of activity of the OAS (preferably OAS1) gene and/or activity of the OAS (preferably OAS1) protein, in the manufacture of a medicament for the treatment of a patient with or at risk of hepatitis C infection, wherein the compound or composition is not an interferon or an isoprenoid, such as geranylgeranylacetone (GGA).

A further aspect of the invention provides for a method of screening for a compound for treating HCV, wherein a cell or isolated OAS (preferably OAS1) protein is treated with a test compound and any change in OAS (preferably OAS1) gene activity and/or OAS (preferably OAS1) protein activity or level is assessed, wherein the test compound is not an interferon or an isoprenoid, such as geranylgeranylacetone (GGA)

A further aspect of the invention provides the use of a compound and/or composition that has the property of modulating the level of activity of the RNAse L gene and/or activity of the RNAse L protein, in the manufacture of a medicament for the treatment of a patient with or at risk of hepatitis C infection, wherein the compound is not an interferon or an isoprenoid, such as geranylgeranylacetone (GGA)

A still futher aspect of the invention provides for a method of screening for compounds for treating HCV, wherein a cell or isolated RNAse L protein is treated with a test compound and any changes in RNAse L gene activity and/or RNAse L protein activity or level is assessed, wherein the test compound is not an interferon or an isoprenoid, such as geranylgeranylacetone (GGA)

A still further aspect of the invention provides for a method of screening for compounds for treating HCV, wherein a cell or isolated 2′-5′ phosphodiesterase protein is treated with a test compound and any changes in 2′-5′ phosphodiesterase gene activity and/or 2′-5′ phosphodiesterase protein activity or level is assessed, wherein the test compound is not an interferon or an isoprenoid, such as geranylgeranylacetone (GGA).

The patient is a human patient with or at risk of exposure to HCV. HCV infection may be indicated by the presence of anti-HCV antibodies and/or HCV RNA (preferably both), as well known to those skilled in the art and, for example, as described in Example 1. The patient may be in a risk group for HCV infection. For example, healthcare workers may be at a greater risk of HCV infection (for example as a consequence of needle stick injuries) than other occupational groups. Intravenous drug users may also be at greater risk. The patient may also be treated with another antiviral therapy or therapies, as well known to those skilled in the art, for example with an interferon, for example an Interferon-α, for example IFN-α2 or IFN-α8.

The activity of the OAS and/or RNAse L and or 2′-5′ phosphodiesterase genes indicated above may be measured by measurement of changes in the activity of the said gene product, for example in a cell, or by techniques such as RT-PCR (reverse transcriptase-polymerase chain reaction), or northern blotting, as well known to those skilled in the art. Alternatively, antibodies to the proteins indicated above may be used to measure the quantity of protein produced in the cell. Proteins may be extracted from a control set of cells and a set of cells after exposure to the potentially modifying compound. The quantity of target protein in the cells may then be determined using methods well known to those skilled in the art, for example western blotting or ELISA.

The effect of the potentially modifying compound on the activity of OAS may be measured using an assay system which may measure the function of the proteins. Methods for measuring the enzyme activity of OAS proteins are disclosed in the literature. For example Sarker and Sen (1998) Methods; 15; 233-242 describe a method of purifying a recombinant OAS protein and assays for protein activity. In another example, Ichikawa et al (2001) Biochem. Biophys. Res. Commun.; 26; 933-939 examine the potential of geranylgeranylacetone (GGA) to modulate OAS gene transcription levels. Hence such methods could form a basis of a screening assay to identify compound that can modulate OAS gene transcription levels and OAS enzyme activity, as will be appreciated by those skilled in the art.

Similarly the effect of the potentially modifying compound on the activity of RNAse L could be measured using an assay system which may measure the enzyme activity. Methods for measuring the enzyme activity of RNAse L are disclosed in the literature. For example, Rusch et al (2001) Methods Enzymol.; 342; 10-20 report a method for measuring the biochemical and biological activity of human RNAse L wherein the enzyme is recombinantly expressed in bacteria and purified and subsequently utilised in an assay method. The same authors also report a method for measuring the biochemical and biological activity of human RNAse L wherein the enzyme activity is measured in intact human cell lines. Hence such methods could form a basis of a screening assay to identify compound that can modulate RNAse L gene transcription levels and RNAse L enzyme activity, as will be appreciated by those skilled in the art.

Similarly the effect of the potentially modifying compound on the activity of 2′-5′ phosphodiesterase could be measured using an assay system which may measure the enzyme activity. Methods for measuring the enzyme activity of 2′-5′ phosphodiesterase are disclosed in the literature. For example, Torrence et al (1983) Anal Biochem; 129; 103-110 report an assay for 2′,5′-oligoadenylate phosphodiesterase activity in mouse L-cell extracts. Such a method could form a basis of a screening assay to identify compound that can modulate 2′-5′ phosphodiesterase gene transcription levels and 2′-5′ phosphodiesterase enzyme activity, as will be appreciated by those skilled in the art.

In a preferred embodiment, test compounds are assessed for their ability to modulate the regulation of RNAseL by OAS and/or 2′-5′ phosphodiesterase. Thus, the test compound may be exposed to a sample comprising both OAS, RNAseL and/or 2′-5′ phosphodiesterase. Further screens (for example on OAS or RNAseL or 2′-5′ phosphodiesterase) alone may be desirable in order to further investigate the mode of action of an active test compound.

It may also be useful to assess test compounds for their ability to modulate the regulation of RNAseL by 2-5A. Thus, it may be preferred to include 2-5A in an assay for RNAseL activity, and/or to perform tests with and without 2-5A which are analysed in parallel.

Methods for performing screening assays will be well known to those skilled in the art. In particular, the effect of different concentrations of the test compound may be determined and a dose response curve calculated. This may allow the effect of the test compound to be expressed in terms of a concentration at which the gene and/or protein activity is 50% of the activity in the absence of the test compound (IC₅₀). However, use of more than one test compound concentration is not essential. A single concentration may be sufficient, particularly in a preliminary screen, for example of test compounds in a test compound bank, as well known to those skilled in the art.

A still further invention provides a compound identified or identifiable by the screening methods of the invention as described above for treating a patient with or a risk of HCV infection, wherein the compound is not an interferon.

A still further aspect of the invention provides the use of a compound identified or identifiable by the screening methods of the invention in the manufacture of a medicament for treating a patient with or at risk of HCV infection.

The compound of the invention is not a compound previously known for treating HCV, for example ribavirin. Ribavirin is a synthetic nucleoside analog that is similar in structure to guanosine that has a broad spectrum of in vitro activity against both DNA and RNA viruses, including Flaviviridae (see, for example, Howard et al (1999) J Hepatol 31 (suppl 1), 152-159).

The compound may be a drug-like compound or lead compound for the development of a drug-like compound for each of the above methods of identifying a compound. It will be appreciated that the said methods may be useful as screening assays in the development of pharmaceutical compounds or drugs, as well known to those skilled in the art.

The term “drug-like compound” is well known to those skilled in the art, and may include the meaning of a compound that has characteristics that may make it suitable for use in medicine, for example as the active ingredient in a medicament. Thus, for example, a drug-like compound may be a molecule that may be synthesised by the techniques of organic chemistry, less preferably by techniques of molecular biology or biochemistry, and is preferably a small molecule, which may be of less than 5000 daltons molecular weight. A drug-like compound may additionally exhibit features of selective interaction with a particular protein or proteins and be bioavailable and/or able to penetrate cellular membranes, but it will be appreciated that these features are not essential.

The binding constant for the binding of the compound to the relevant polypeptide may be determined. Suitable methods for detecting and/or measuring (quantifying) the binding of a compound to a polypeptide are well known to those skilled in the art and may be performed, for example using a method capable of high throughput operation, for example a chip-based method in which the compounds to be tested are immobilised in a microarray on a solid support, as known to those skilled in the art.

A further aspect of the invention provides the use of a nucleic acid which hybridises selectively to a OAS, preferably OAS1 nucleic acid, for example the OAS1 gene, in the manufacture of a medicament for the treatment of a patient with or a risk of HCV infection. A further aspect of the invention provides the use of a nucleic acid which hybridises selectively to a OAS, preferably OAS1 nucleic acid, for example the OAS1 gene, in the manufacture of a diagnostic reagent for use in the assessment or diagnosis of a patient with or a risk of HCV infection.

A further aspect of the invention provides the use of a OAS polypeptide (preferably OAS1) in the manufacture of a medicament for the treatment of a patient with or at risk of HCV infection.

The patient is a human patient with or at risk of exposure to HCV. The reagent may be useful in assessing the patient in order to decide on appropriate treatment for the patient. For example, the assessment may be useful in determining whether the patient has a high or low risk of developing a persistent infection or adverse consequences (such as fibrosis) of infection, and therefore whether the patient should be administered antiviral therapy at an early stage (if the risk is high) or should be monitored for longer before beginning such therapy (if the risk is low, thereby avoiding unnecessary side-effects and costs).

The method may be used as an adjunct to known assessment or prognostic methods such as histopathological examination of biopsy tissue, or imaging or serum marker assays (Imbert-Bismut F et al (2001) Biochemical markers of liver fibrosis in patients with hepatitis C virus infection: a prospective study. Lancet 357(9262):1069-1075; Rosenberg W et al (2001) Serum Markers Predict Liver Fibrosis. British Association for the study of the Liver Meeting 2001, Book of Abstracts, 23). The method may also be used in conjunction with consideration of other risk factors, for example sex (males may be at higher risk of rapid fibrosis) and age at infection (higher age at infection may be associated with a higher risk of rapid fibrosis). Other parameters used to measure HCV infection include assessing viral genotype and viral load (for example, Murashima et al (2000) J. Med Virol 62 185-190). It is preferred in relation to treatment that the patient to be treated has a GG genotype at position 84 bp of the untranslated 3′ end of exon 8 as shown in FIG. 1 and discussed in Example 1.

By “hybridises selectively” is meant that the nucleic acid has sufficient nucleotide sequence similarity with the said human nucleic acid that it can hybridise under moderately or highly stringent conditions, and preferably does not hybridise to other (non-OAS) nucleic acids under the same conditions. Preferably, it does not hybridise to OAS2 or OAS3 nucleic acid (ie OAS2 or OAS3 genes, mRNA or cDNA). As is well known in the art, the stringency of nucleic acid hybridization depends on factors such as length of nucleic acid over which hybridisation occurs, degree of identity of the hybridising sequences and on factors such as temperature, ionic strength and CG or AT content of the sequence. Thus, any nucleic acid which is capable of selectively hybridising as said is useful in the practice of the invention.

Nucleic acids which can selectively hybridise to the said human nucleic acid include nucleic acids which have >95% sequence identity, preferably those with >98%, more preferably those with >99% sequence identity, over at least a portion of the nucleic acid with the said human nucleic acid. As is well known, human genes usually contain introns such that, for example, a mRNA or cDNA derived from a gene would not match perfectly along its entire length with the said human genomic DNA but would nevertheless be a nucleic acid capable of selectively hybridising to the said human DNA. Thus, the invention specifically includes nucleic acids which selectively hybridise to said OAS1 (or OAS2 or OAS3) mRNA or cDNA but may not hybridise to said OAS1 (or OAS2 or OAS3) gene.

Typical moderately or highly stringent hybridisation conditions which lead to selective hybridisation are known in the art, for example those described in Molecular Cloning, a laboratory manual, 2nd edition, Sambrook et al (eds), Cold Spring Harbor Laboratory Press, Cold Spring Harbor, N.Y., USA, incorporated herein by reference.

An example of a typical hybridisation solution when a nucleic acid is immobilised on a nylon membrane and the probe nucleic acid is ≧500 bases is:

6×SSC (saline sodium citrate)

0.5% sodium dodecyl sulphate (SDS)

100 μg/ml denatured, fragmented salmon sperm DNA

The hybridisation is performed at 68° C. The nylon membrane, with the nucleic acid immobilised, may be washed at 68° C. in 1×SSC or, for high stringency, 0.1×SSC.

20×SSC may be prepared in the following way. Dissolve 175.3 g of NaCl and 88.2 g of sodium citrate in 800 ml of H₂O. Adjust the pH to 7.0 with a few drops of a 10 N solution of NaOH. Adjust the volume to 1 litre with H₂O. Dispense into aliquots. Sterilise by autoclaving.

An example of a typical hybridisation solution when a nucleic acid is immobilised on a nylon membrane and the probe is an oligonucleotide of between 15 and 50 bases is:

3.0 M trimethylammonium chloride (TMACl)

0.01 M sodium phosphate (pH 6.8)

1 mm EDTA (pH 7.6)

0.5% SDS

100 μg/ml denatured, fragmented salmon sperm DNA

0.1% non-fat dried milk

The optimal temperature for hybridization is usually chosen to be 5° C. below the Tᵢ for the given chain length. Tᵢ is the irreversible melting temperature of the hybrid formed between the probe and its target sequence. Jacobs et al (1988) Nucl. Acids Res. 16, 4637 discusses the determination of Tᵢs. The recommended hybridization temperature for 17-mers in 3 M TMACl is 48-50° C.; for 19-mers, it is 55-57° C.; and for 20-mers, it is 58-66° C.

By “nucleic acid which selectively hybridises” is also included nucleic acids which may be used to amplify DNA from the OAS1 (or OAS2 or OAS3) gene or cDNA (for example formed by reverse transcription of mRNA) by any of the well known amplification systems such as those described in more detail below, in particular the polymerase chain reaction (PCR).

Suitable conditions for PCR amplification include amplification in a suitable 1× amplification buffer:

10× amplification buffer is 500 mM KCl; 100 mM Tris.Cl (pH 8.3 at room temperature); 15 mM MgCl₂; 0.1% gelatin.

A suitable denaturing agent or procedure (such as heating to 95° C.) is used in order to separate the strands of double-stranded DNA.

Suitably, the annealing part of the amplification is between 37° C. and 60° C., preferably 50° C.

As stated above it is preferred that the patient to be treated has a GG genotype 84 bp into the untranslated 3′ end of exon 8 as shown in FIG. 1.

Methods of genotypic analysis will be well known to those skilled in the art. The genotype may preferably be determined by testing a sample from the patient. Preferably, the sample contains nucleic acid, such as genomic DNA or mRNA (preferably genomic DNA), and the genotype is determined by a method which involves contacting said nucleic acid (or nucleic acid derived from it, for example cDNA when the target nucleic acid is an RNA, for example a mRNA) with a nucleic acid which hybridises selectively to said OAS1 nucleic acid.

For example, genotyping may be performed by any one of a large number of assays, for example sequencing, RFLP, ARMS PCR, PCR and sequence-specific oligonucleotide hybridisation, SnapShot PCR, Ligase detection reaction, PCR and Maldi-TOF, Pyrosequencing. These and other suitable techniques will be well known to those skilled in the art.

The sample may conveniently be whole blood or genomic DNA extracted from whole blood, for example a 5 ml sample collected into EDTA and extracted using a commercial kit (for example as supplied by Nucleon II, Scotlabs, UK).

By “selectively hybridising” is meant that the nucleic acid has sufficient nucleotide sequence similarity with the said (human) nucleic acid that it can hybridise under moderately or highly stringent conditions, as discussed above.

It is preferred that the method of identifying the genotype at position 84 of the OAS1 gene of a patient is based on allele specific PCR, a technique well known to those skilled in the art. The method employed in the present study is described in detail in Example 1.

A further aspect of the invention provides one or more oligonucleotide primers (for example a pair or series) suitable for use in determining the sequence of the OAS1 gene 84 bp into the untranslated 3′ end of exon 8 (for example as used in the method described in Example 1) for use in medicine.

A further aspect of the invention provides the use of said one or more oligonucleotide primers in the manufacture of a kit or reagent for use in the diagnosis or assessment of a patient with or at risk of HCV. The reagent or kit may comprise two or more oligonucleotide primers suitable for use in an amplification reaction (for example a PCR) for amplifying sequence including the defined position; thus the primers may flank the defined position (preferably each within 50, 100, 200 or 500 bases thereof) hybridise to opposite strands of the OAS nucleic acid, as well known to those skilled in the art.

Although any sample containing nucleic acid derived from the patient is useful in the methods of the invention when determining genotype, it is preferred if the sample is readily obtainable from the patient, for example blood, semen or skin cells (for example a sample of cells from the buccal cavity). Liver tissue taken at biopsy may be used. Most conveniently the sample is blood. Although it is preferred that the sample containing nucleic acid from the patient is, or is derived directly from, a cell of the patient, a sample indirectly derived from a patient, such as a cell grown in culture, is also included within the invention. Equally, although the nucleic acid derived from the patient may have been physically within the patient, it may alternatively have been copied from nucleic acid which was physically within the patient.

Conveniently, the nucleic acid capable of selectively hybridising to the said human nucleic acid such as genomic DNA and which is used in the methods of the invention may further comprise a detectable label.

By “detectable label” is included any convenient radioactive label such as ³²P, ³³P or ³⁵S which can readily be incorporated into a nucleic acid molecule using well known methods; any convenient fluorescent or chemiluminescent label which can readily be incorporated into a nucleic acid is also included. In addition the term “detectable label” also includes a moiety which can be detected by virtue of binding to another moiety (such as biotin which can be detected by binding to streptavidin); and a moiety, such as an enzyme, which can be detected by virtue of its ability to convert a colourless compound into a coloured compound, or vice versa (for example, alkaline phosphatase can convert colourless o-nitrophenylphosphate into coloured o-nitrophenol). Conveniently, the nucleic acid probe may occupy a certain position in a fixed array and whether the nucleic acid hybridises to the said coagulation factor nucleic acid can be determined by reference to the position of hybridisation in the fixed array.

Primers which are suitable for use in a polymerase chain reaction (PCR; Saiki et al (1988) Science 239, 487-491) are preferred. Suitable PCR primers may have the following properties:

It is well known that the sequence at the 5′ end of the oligonucleotide need not match the target sequence to be amplified.

It is usual that the PCR primers do not contain any complementary structures with each other longer than 2 bases, especially at their 3′ ends, as this feature may promote the formation of an artifactual product called “primer dimer”. When the 3′ ends of the two primers hybridize, they form a “primed template” complex, and primer extension results in a short duplex product called “primer dimer”.

Internal secondary structure should be avoided in primers. For symmetric PCR, a 40-60% G+C content is often recommended for both primers, with no long stretches of any one base. The classical melting temperature calculations used in conjunction with DNA probe hybridization studies often predict that a given primer should anneal at a specific temperature or that the 72° C. extension temperature will dissociate the primer/template hybrid prematurely. In practice, the hybrids are more effective in the PCR process than generally predicted by simple Tₘ calculations.

Optimum annealing temperatures may be determined empirically and may be higher than predicted. Taq DNA polymerase does have activity in the 37-55° C. region, so primer extension will occur during the annealing step and the hybrid will be stabilized. The concentrations of the primers are equal in conventional (symmetric) PCR and, typically, within 0.1- to 1 μM range.

Any of the nucleic acid amplification protocols can be used in the method of the invention including the polymerase chain reaction, QB replicase and ligase chain reaction. Also, NASBA (nucleic acid sequence based amplification), also called 3SR, can be used as described in Compton (1991) Nature 350, 91-92 and AIDS (1993), Vol 7 (Suppl 2), S108 or SDA (strand displacement amplification) can be used as described in Walker et al (1992) Nucl. Acids Res. 20, 1691-1696. The polymerase chain reaction is particularly preferred because of its simplicity.

When a pair of suitable nucleic acids of the invention are used in a PCR it is possible to detect the product by gel electrophoresis and ethidium bromide staining. As an alternative to detecting the product of DNA amplification using agarose gel electrophoresis and ethidium bromide staining of the DNA, it is convenient to use a labelled oligonucleotide capable of hybridising to the amplified DNA as a probe. When the amplification is by a PCR the oligonucleotide probe hybridises to the interprimer sequence as defined by the two primers. The oligonucleotide probe is preferably between 10 and 50 nucleotides long, more preferably between 15 and 30 nucleotides long. The probe may be labelled with a radionuclide such as ³²P, ³³P and ³⁵S using standard techniques, or may be labelled with a fluorescent dye. When the oligonucleotide probe is fluorescently labelled, the amplified DNA product may be detected in solution (see for example Balaguer et al (1991) “Quantification of DNA sequences obtained by polymerase chain reaction using a bioluminescence adsorbent” Anal. Biochem. 195, 105-110 and Dilesare et al (1993) “A high-sensitivity electrochemiluminescince-based detection system for automated PCR product quantitation” BioTechniques 15, 152-157.

PCR products can also be detected using a probe which may have a fluorophore-quencher pair or may be attached to a solid support or may have a biotin tag or they may be detected using a combination of a capture probe and a detector probe.

Fluorophore-quencher pairs are particularly suited to quantitative measurements of PCR reactions (eg RT-PCR). Fluorescence polarisation using a suitable probe may also be used to detect PCR products.

It is preferred for therapeutic purposes that the polynucleotide comprises the ‘A’ genotype OAS1 gene or portions thereof encompassing the DNA sequence 84 bp into the untranslated 3′ end of exon 8 as shown in FIG. 1.

The polynucleotide encoding the ‘A’ genotype OAS1 gene (ie, the polynucleotide has an A nucleotide residue 84 bp into the untranslated 3′ end of exon 8 as shown in FIG. 1) may be prepared using techniques well known to those skilled in the art and as described below. Preferably the polynucleotide is capable of expressing the OAS1 polypeptide in the patient. The said polypeptide may be expressed from any suitable polynucleotide (genetic construct) as is described below and delivered to the patient. Typically, the genetic construct which expresses the polypeptide comprises the said polypeptide coding sequence operatively linked to a promoter which can express the transcribed polynucleotide (eg mRNA) molecule in a cell of the patient, which may be translated to synthesise the said polypeptide. Suitable promoters will be known to those skilled in the art, and may include promoters for ubiquitously expressed, for example housekeeping genes or for tissue-specific genes, depending upon where it is desired to express the said polypeptide, as discussed further below.

Hence a further aspect of the invention provides a polynucleotide vector comprising a polynucleotide sequence as defined in FIG. 1 and detailed in the previous sections of the document. Preferably the vector comprising the polynucleotide sequence is suitable for expressing OAS1 in a target cell of the patient.

Although the genetic construct can be DNA or RNA it is preferred if it is DNA.

Preferably, the genetic construct is adapted for delivery to a human cell.

Means and methods of introducing a genetic construct into a cell in an animal body are known in the art. For example, the constructs of the invention may be introduced into the cells by any convenient method, for example methods involving retroviruses, so that the construct is inserted into the genome of the (dividing) cell. Targeted retroviruses are available for use in the invention; for example, sequences conferring specific binding affinities may be engineered into pre-existing viral env genes (see Miller & Vile (1995) Faseb J. 9, 190-199 for a review of this and other targeted vectors for gene therapy).

It will be appreciated that retroviral methods, such as those described below, may only be suitable when the cell is a dividing cell. For example, in Kuriyama et al (1991) Cell Struc. and Func. 16, 503-510 purified retroviruses are administered. Retroviral DNA constructs which encode the said polypeptide may be made using methods well known in the art. To produce active retrovirus from such a construct it is usual to use an ecotropic psi2 packaging cell line grown in Dulbecco's modified Eagle's medium (DMEM) containing 10% foetal calf serum (FCS). Transfection of the cell line is conveniently by calcium phosphate co-precipitation, and stable transformants are selected by addition of G418 to a final concentration of 1 mg/ml (assuming the retroviral construct contains a neo® gene). Independent colonies are isolated and expanded and the culture supernatant removed, filtered through a 0.45 μm pore-size filter and stored at −70° C. For the introduction of the retrovirus into the target cells, it is convenient to inject directly retroviral supernatant to which 10 μg/ml Polybrene has been added. The injection may be made into the area in which the target cells are present. It may be desirable to express the antigenic polypeptides in antigen presenting cells (APCs).

Other methods involve simple delivery of the construct into the cell for expression therein either for a limited time or, following integration into the genome, for a longer time. An example of the latter approach includes liposomes (Nissander et al (1992) Cancer Res. 52, 646-653). Other methods of delivery include adenoviruses carrying external DNA via an antibody-polylysine bridge (see Curiel Prog. Med. Virol. 40, 1-18) and transferrin-polycation conjugates as carriers (Wagner et al (1990) Proc. Natl. Acad. Sci. USA 87, 3410-3414). The DNA may also be delivered by adenovirus wherein it is present within the adenovirus particle. It will be appreciated that “naked DNA” and DNA complexed with cationic and neutral lipids may also be useful in introducing the DNA of the invention into cells of the patient to be treated. Non-viral approaches to gene therapy are described in Ledley (1995) Human Gene Therapy 6, 1129-1144. Alternative targeted delivery systems are also known such as the modified adenovirus system described in WO 94/10323 wherein, typically, the DNA is carried within the adenovirus, or adenovirus-like, particle. Michael et al (1995) Gene Therapy 2, 660-668 describes modification of adenovirus to add a cell-selective moiety into a fibre protein. Mutant adenoviruses which replicate selectively in p53-deficient human tumour cells, such as those described in Bischoff et al (1996) Science 274, 373-376 are also useful for delivering the genetic construct to a cell. Other suitable viruses or virus-like particles include HSV, AAV, vaccinia and parvovirus.

Immunoliposomes (antibody-directed liposomes) are especially useful in targeting to cell types which over-express a cell surface protein for which antibodies are available. For the preparation of immuno-liposomes MPB-PE (N-[4-(p-maleimidophenyl)butyryl]-phosphatidylethanolamine) is synthesised according to the method of Martin & Papahadjopoulos (1982) J. Biol. Chem. 257, 286-288. MPB-PE is incorporated into the liposomal bilayers to allow a covalent coupling of the antibody, or fragment thereof, to the liposomal surface. The liposome is conveniently loaded with the genetic construct for delivery to the target cells, for example, by forming the said liposomes in a solution of the genetic construct, followed by sequential extrusion through polycarbonate membrane filters with 0.6 μm and 0.2 μm pore size under nitrogen pressures up to 0.8 MPa. After extrusion, entrapped DNA construct is separated from free DNA construct by ultracentrifugation at 80 000×g for 45 min. Freshly prepared MPB-PE-liposomes in deoxygenated buffer are mixed with freshly prepared antibody (or fragment thereof) and the coupling reactions are carried out in a nitrogen atmosphere at 4° C. under constant end over end rotation overnight. The immunoliposomes are separated from unconjugated antibodies by ultracentrifugation at 80 000×g for 45 min. Immunoliposomes may be injected, for example intraperitoneally or directly into a site where the target cells are present.

Other methods of delivery include adenoviruses carrying external DNA via an antibody-polylysine bridge (see Curiel Prog. Med. Virol. 40, 1-18) and transferrin-polycation conjugates as carriers (Wagner et al (1990) Proc. Natl. Acad. Sci. USA 87, 3410-3414). In the first of these methods a polycation-antibody complex is formed with the genetic construct, wherein the antibody is specific for either wild-type adenovirus or a variant adenovirus in which a new epitope has been introduced which binds the antibody. The polycation moiety binds the DNA via electrostatic interactions with the phosphate backbone. The adenovirus, because it contains unaltered fibre and penton proteins, is internalised into the cell and carries into the cell with it the DNA construct of the invention. It is preferred if the polycation is polylysine.

DNA may also be delivered by adenovirus wherein it is present within the adenovirus particle, for example, as described below.

In the second of these methods, a high-efficiency nucleic acid delivery system that uses receptor-mediated endocytosis to carry DNA macromolecules into cells is employed. This is accomplished by conjugating the iron-transport protein transferrin to polycations that bind nucleic acids. Human transferrin, or the chicken homologue conalbumin, or combinations thereof is covalently linked to the small DNA-binding protein protamine or to polylysines of various sizes through a disulfide linkage. These modified transferrin molecules maintain their ability to bind their cognate receptor and to mediate efficient iron transport into the cell. The transferrin-polycation molecules form electrophoretically stable complexes with DNA constructs or other genetic constructs independent of nucleic acid size (from short oligonucleotides to DNA of 21 kilobase pairs). When complexes of transferrin-polycation and the DNA constructs or other genetic constructs are supplied to the target cells, a high level of expression from the construct in the cells is expected.

High-efficiency receptor-mediated delivery of the DNA constructs or other genetic constructs using the endosome-disruption activity of defective or chemically inactivated adenovirus particles produced by the methods of Cotten et al (1992) Proc. Natl. Acad. Sci. USA 89, 6094-6098 may also be used. This approach appears to rely on the fact that adenoviruses are adapted to allow release of their DNA from an endosome without passage through the lysosome, and in the presence of, for example transferrin linked to the genetic construct, the construct is taken up by the cell by the same route as the adenovirus particle.

This approach has the advantages that there is no need to use complex retroviral constructs; there is no permanent modification of the genome as occurs with retroviral infection; and the targeted expression system is coupled with a targeted delivery system, thus reducing toxicity to other cell types.

It may be desirable to locally perfuse an area comprising target cells with the suitable delivery vehicle comprising the genetic construct for a period of time; additionally or alternatively the delivery vehicle or genetic construct can be injected directly into accessible areas comprising target cells. It may be beneficial to deliver the delivery vehicle or genetic construct systemically.

The genetic constructs can be prepared using methods well known in the is art.

It will be appreciated that it may be desirable to be able to regulate temporally expression of the said polypeptide in the cell. Thus, it may be desirable that expression of the said polypeptide is directly or indirectly (see below) under the control of a promoter that may be regulated, for example by the concentration of a small molecule that may be administered to the patient when it is desired to activate or repress (depending upon whether the small molecule effects activation or repression of the said promoter) expression of the said polypeptide. It will be appreciated that this may be of particular benefit if the expression construct is stable ie capable of expressing the said polypeptide (in the presence of any necessary regulatory molecules) in the said cell for a period of at least one week, one, two, three, four, five, six, eight months or one or more years. A preferred construct may comprise a regulatable promoter. Examples of regulatable promoters include those referred to in the following papers: Rivera et al (1999) Proc Natl Acad Sci USA 96(15), 8657-62 (control by rapamycin, an orally bioavailable drug, using two separate adenovims or adeno-associated virus (AAV) vectors, one encoding an inducible human growth hormone (hGH) target gene, and the other a bipartite rapamycin-regulated transcription factor); Magari et al (1997) J Clin Invest 100(11), 2865-72 (control by rapamycin); Bueler (1999) Biol Chem 380(6), 613-22 (review of adeno-associated viral vectors); Bohl et al (1998) Blood 92(5), 1512-7 (control by doxycycline in adeno-associated vector); Abruzzese et al (1996) J Mol Med 74(7), 379-92 (reviews induction factors e.g., hormones, growth factors, cytokines, cytostatics, irradiation, heat shock and associated responsive elements). Tetracycline—inducible vectors may also be used. These are activated by a relatively non-toxic antibiotic that has been shown to be useful for regulating expression in mammalian cell cultures. Also, steroid-based inducers may be useful especially since the steroid receptor complex enters the nucleus where the DNA vector must be segregated prior to transcription.

This system may be further improved by regulating the expression at two levels, for example by using a tissue-specific promoter and a promoter controlled by an exogenous inducer/repressor, for example a small molecule inducer, as discussed above and known to those skilled in the art. Thus, one level of regulation may involve linking the appropriate sequence encoding the polypeptide to an inducible promoter whilst a further level of regulation entails using a tissue-specific promoter to drive the gene encoding the requisite inducible transcription factor (which controls expression of the polypeptide from the inducible promoter). The tissue-specific promoter may be a liver-specific promoter, for example the elongation factor α (EF1-α) promoter. Other liver-specific promoters include the albumin promoter and the transthyretin promoter (Quian et al (1995) Mol Cell Biol 15, 1364-1376; Bristol J A, Gallo-Penn A, Andrews J, Idamakanti N, Kaleko M, Connelly S. (2001) Hum Gene Ther vol 12(13):1651-61). Control may further be improved by cell-type-specific targeting of the genetic construct.

A further aspect of the invention provides the use of an OAS1 polynucleotide or compound of the invention that modifies the activity of the OAS1 and/or RNAse L and/or 2′-5′ phosphodiesterase genes and/or proteins as described above in the manufacture of a medicament for the treatment of a patient with or at risk of hepatitis C infection.

A further aspect of the invention provides a method of treating a patient with or at risk of hepatitis C infection, wherein the patient is administered an OAS1 polynucleotide or compound of the invention. It is preferred that the patient is of OAS genotype GG as described above.

The aforementioned compounds or a formulation thereof may be administered by any conventional method including oral and parenteral (eg subcutaneous or intramuscular) injection. The treatment may consist of a single dose or a plurality of doses over a period of time.

Whilst it is possible for a compound to be administered alone, it is preferable to present it as a pharmaceutical formulation, together with one or more acceptable carriers. The carrier(s) must be “acceptable” in the sense of being compatible with the compound and not deleterious to the recipients thereof. Typically, the carriers will be water or saline which will be sterile and pyrogen free.

The formulations may conveniently be presented in unit dosage form and may be prepared by any of the methods well known in the art of pharmacy. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations in accordance with the present invention suitable for oral administration may be presented as discrete units such as capsules, cachets or tablets, each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder (eg povidone, gelatin, hydroxypropylmethyl cellulose), lubricant, inert diluent, preservative, disintegrant (eg sodium starch glycolate, cross-linked povidone, cross-linked sodium carboxymethyl cellulose), surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethylcellulose in varying proportions to provide desired release profile.

Formulations suitable for topical administration in the mouth include lozenges comprising the active ingredient in a flavoured basis, usually sucrose and acacia or tragacanth; pastilles comprising the active ingredient in an inert basis such as gelatin and glycerin, or sucrose and acacia; and mouth-washes comprising the active ingredient in a suitable liquid carrier.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit, daily sub-dose or an appropriate fraction thereof, of an active ingredient.

It should be understood that in addition to the ingredients particularly mentioned above the formulations of this invention may include other agents conventional in the art having regard to the type of formulation in question, for example those suitable for oral administration may include flavouring agents.

A further aspect of the invention provides a pharmaceutical composition or a kit of parts comprising (1) (a) a compound that is capable of modulating the level of activity of the OAS1 gene and/or activity of the OAS1 protein, and/or (b) a compound that is capable of modulating the level of activity of the RNAse L gene and/or activity of the RNAse L protein, and/or (c) a compound that is capable of modulating the level of activity of the 2′-5′ phosphodiesterase gene and/or activity of the 2′-5′ phosphodiesterase protein, and/or (d) a recombinant polynucleotide or gene therapy vector of the invention, and (2) a therapeutically appropriate quantity of an interferon, for example an interferon-α, for example interferon-α8, and optionally (3) a pharmaceutically acceptable diluent or carrier (for example sterile saline or water). The pharmaceutical composition may be suitable for presentation to the patient as a pharmaceutical formulation as described above. The compound may preferably be a compound of the invention.

A further aspect of the invention provides a kit of parts comprising (1) a pharmaceutical composition or kit as described above and (2) reagents suitable for identifying the OAS1 genotype of a patient with or at risk of infection with HCV.

All documents referred to herein are hereby incorporated by reference.

The invention is now described in more detail by reference to the following, non-limiting Examples and Figures.

FIG. 1: Sequence listing of the OAS1 gene

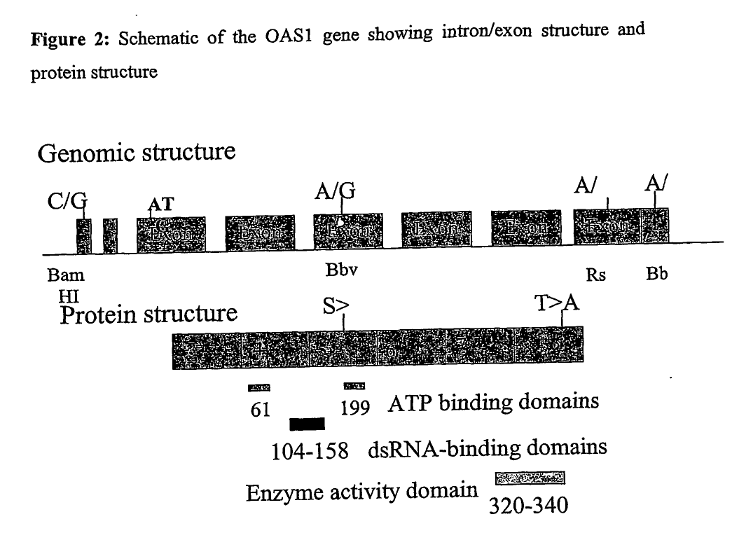

FIG. 2: Schematic of the OAS1 gene showing intron/exon structure and protein structure

### EXAMPLE 1

### Association of Polymorphisms in OAS1 with Self Limiting HCV Infection

Introduction

A small proportion of patients infected with the hepatitis C virus (HCV) will spontaneously eliminate the virus. It has been postulated that host genetic factors influence resistance to persistent infection and the outcome of treatment. Among the candidate genes is the interferon-induced gene 2′,5′-oligoadenylate synthetase, which has an important role in the viral defence mechanism. Upon binding to dsRNA, OAS catalyses the formation of 2′,5′ linked oligoadenylate and activates RNAse L, which breaks down viral and cellular RNA. However, it appears that OAS has no function in the action of interferon-α during HCV therapy and, overall, the role of OAS in the response of a patient to HCV is very unclear. In contrast, interferon-α induced RNA-dependant protein kinase (PKR) and and myxovirus (influenza) resistance 1 (MxA) genes do play a role in the elimination of HCV in patients treated with interferon-α a (Chieux et al (1998) J. Virol. Methods; 70; 183-191; Yu et al (2000) Hepatology; 32; 1089-1095).

We set out to examine whether there were polymorphisms in PKR, MxA and OAS which were associated with the efficiency with which the HCV is eliminated from a patient.

Methods

Subjects

The study subjects were recruited at nine large hepatology units distributed across Europe between October 1995 and June 2001. Informed consent was obtained from each subject and the study was approved by the local research ethics committee in each centre. Subjects with self limiting infection had antibodies to HCV (ELISA positive and RIBA positive or RIBA indeterminate), and were negative for viraemia on all occasions after presentation. Patients were tested for viraemia at the time of presentation and on at least one other occasion three months or more after presentation. These subjects had consistently normal liver transaminases (alanine transaminase and/or aspartate transaminase less than or equal to the upper limit of normal); samples were taken at least twice over a three month period. Subjects with persistent viral infection were HCV antibody positive and PCR positive for at least six months or at least six months after the likely exposure to HCV. A control group of 132 healthy subjects was recruited to establish the allele frequencies of the trinucleotide polymorphism in the PKR promoter.

The presence or absence of viral particles in serum was determined by reverse transcription polymerase chain reaction using a commercially available assay (Amplicor, Roche) with a sensitivity of approximately 200 genomes/ml. Antibodies (Abbot, Maidenhead, U.K) to HCV antigens were detected with both an enzyme linked immunoassay EIA or a recombinant immunoblot assay RIBA containing 4 HCV antigens on a cellulose acetate strip used according to the manufacturer's instructions.

SNP's

Information of SNP's was taken (from literature), public database or sequencing.

Sequencing

PKR:

It was calculated that an allele with a frequency of 0.2 or greater in the general population would be detected with 95% probability if 25 haplotypes were examined. We therefore decided to sequence 15 normal individuals, representing 30 haplotypes, in order to detect novel polymorphisms in the PKR gene.

Using the published primary sequence of the PKR gene primers were designed for PCR amplification of the promoter and 5′-untranslated region, exons 4 & 6 (representing the RNA binding domain), exon 11 (encoding the NS5a binding site) and exons 11, 12, 13 (encoding the kinase catalytic domain) (Gale et al (1997) Virology; 230; 217-227; Feng et al (1992) Proc. Natl. Acad. Sci. USA; 89; 5447-551; McMillan et al (1996) in Protein phosphorylation in cell growth regulation; London, Harwood) and exon 17, the largest coding exon. The primer sequences listed in Table 1.

PCR products for the functional domains were obtained using the following reaction conditions: 100-200 ng of genomic DNA were added to PCR buffer (Qiagen), 0.2 units Taq polymerase (Qiagen), 200 uM dNTPs and 2.5 μM of each primer in a total volume of 20 μL with a final concentration of 1.5 mM MgCl₂. PCR reactions were performed on a Perkin Elmer 2400 thermal cycler. After an initial 5 minutes at 94° C., 35 cycles were conducted at 94° C. for 30 seconds, 55° C. for 30 seconds and 72° C. for 1 minute, with a final extension at 72° C. for 7 minutes.

PCR conditions for the 5′ untranslated region, promoter and exon 1 had to be modified as they are highly GC rich. 100-200 ng of genomic DNA was added to PCR buffer (Qiagen), Q buffer (Qiagen), 1.5 μM MgCl₂ (final concentration), 1 unit Taq polymerase, 200 μM dNTPs and 2.5 μM of each primer in a total volume of 20 μl. PCR reactions were performed on a Perkin Elmer 2400 thermal cycler. After an initial 5 minutes at 94° C., 35 cycles were conducted at 94° C. for 30 seconds, 68° C. for 30 seconds and 72° C. for 1 minute with a final extension at 72° C. for 7 minutes.

The presence of PCR products was confirmed by running 2 μl of product on a 2% agarose gel in TAE buffer. 10 μl of PCR product was then run on a 2% agarose gel and the product band was extracted and purified (Qiagen Gel Extraction Kit). 5 μl of this purified PCR product was used as a template for sequencing reactions which were carried out using the Big Dye Terminator kit (Applied Biosystems) according to the manufacturers instructions. Sequencing was carried out on a ABI 377 automated sequencer (Applied Biosystems) and analysed using Sequence Analysis software 2.1 (Applied Biosystems).

MxA

Two primer pairs were designed to produce fragments for sequencing covering up to 1.5 kb from the transcription start site. MxAp1: 5′-ggcctggcctgacaactat-3′ and MxAp1R: 5′-catccaagcctgcacgtat (product size 403 bp) and MxAp2F: 5′-gctttgtgtgagcaacatgg and MXAp2R: 5′-ggctcatctggtctctccag (product size 523 bp).

OAS-1:

Sequencing of functional regions of OAS-1 was carried out in order to confirm SNP's identified through database searches.

Genotyping

PKR:

Variation in the number of CGG repeats in the first exon/5′ untranslated region of PKR was determined by fluorescent labelled product size discrimination using the ABI 377 automated sequencer/genotyper. PCR products were obtained using the PCR method used for sequencing described above with a FAM-labelled sense primer. The products were diluted between 1:5 and 1:20 in water and 1 μl of the diluted product was added to 4 μl of formamide, TAMRA 350 size markers and Bluedextran/EDTA buffer (ratio 3:1:1). The samples were heated to 94° C. for 5 minutes and then cooled on ice immediately. 2 μl of this solution was loaded onto a denaturing 4.25% polyacrylamide gel and run on an ABI 377 genotyping system. Allele discrimination was performed using Genescan and Genotyper software.

Point mutations in the promoter region of PKR were genotyped by sequence analysis. In addition restriction fragment length polymorphism (RFLP) with SgrAI was used to genotype the SNP at position 486 (167 bp from the transcription start) of the PKR gene. On this occasion a 282 bp fragment was amplified using the forward primer 5′-gactaggccagcggagaac-3′ and the reverse primer 5′-gcttcgggagagctggtt-3′. In the presence of the T allele a second SgrAI site was introduced which leads to the separation of a 19, 97 and 166 bp fragments after digestion and gel separation, whereas in presence of the T allele the SgrAI digest only gives rise to a 116 and 166 bp fragment.

MxA:

The biallelic G/T polymorphism in the promoter region of MxA at position −88 from the transcription start site (published by Hijikata et al, 2000) was genotyped by RFLP using HhaI (New England Biolabs). The primers 5′-tgaagacccccaattaccaa-3′ and 5′-ctctcgttcgcctctttcac-3′ were used to amplify a fragment of 351 bp. Amplification was carried out in a volume of 20 μl, containing 10-100 ng DNA, 2.5 mM MgCl2, 500 nM of each primer, 500 μM dNTP's, 1×PCR buffer (Qiagen), 1 unit Taq DNA polymerase (Qiagen), 0.16 μl TaqStart Antibody (BD Clontech). The cycling conditions in an Applied Biosystems 2400 or 9700 machine were: denaturation at 94° C. for 5 minutes, subsequently 35 cycles of denaturation at 94° C. for 30 seconds; annealing at 58° C. for 30 seconds; and extension at 72° C. for 1 minute. This was followed by a final extension step at 72° C. for 7 minutes. For the HhaI restriction digest 8 μl of the PCR product were digested for at least 4 hours or over night in a volume of 20 μl and 5 units of HhaI according to manufacturers specifications. 10 μl of the digested PCR product were run out on 2% agarose gels and analysed. In the presence of the G allele the 351 bp long product is cut into 260, 51, 23 and 16 bp. A SNP (G/A) at position −168 from the transcription start site was genotyped using BsaI (New England Biolabs) to differentiate between the alleles. A 402 bp fragment was amplified at the conditions previously described, using primers 5′-ggcctggcctgacaactat-3′ and 5′-catccaagcctgcacgtat-3′. Restriction digestion was carried out using 8 μl of the PCR product in 20 μl volume in the presence of 2 units BsaI. Presence of the A-allele was indicated by the absence of the BsaI cutting site, whereas the G allele results in the creation of a 287 bp and a 115 bp fragment.

OAS-1:

The A/G SNP at position 84 bp in the untranslated 3′end of exon 8 of OAS1, (position 347 of genebank accession number M11810) was genotyped using allele specific PCR on an Applied Biosystems 5700 machine. In this method each reaction comprised 0.2 μM of each of the following primers: either 5′-CTCACTGAGGAGCTTTGTCT-3′ or 5′-CACTGAGGAGCTTTGTCC-3′ (reverse A/G-allele specific primers, and 5′-CAGGTGGGACTCTTGATCCAG-3′ (forward common primer); 2.5 units of Stoffel Gold Polymerase (David Birch, RMS); 1× Stoffel Gold buffer (10 mM Tris-HCl, 10 mM KCL at pH 8.0); an additional 30 mM KCl for a final concentration of 40 mM; 2 mM MgCl2; 50 μM each dATP, dCTP, and dGTP; 25 mM TTP; 75 mM dUTP: 2 units of UNG (PE), 0.2× SybrGreen I (Roche Molecular Probes); 2 μM ROX (Roche Molecular Probes); 5% DMSO; and 2.5% Glycerol.

Kinetic PCR reactions were performed on a GeneAmp 5700 Sequence detection System (PE Applied Biosystems). An initial incubation step of 2 min at 50° C., (to allow UNG mediated elimination of carryover PCR product contamination), and an enzyme heat activation step of 12 min at 95° C. were followed by 40 two-step amplification cycles of 20 sec at 95° C. for denaturation and 20 sec for 58° C. for annealing and extension, and a final 5 minutes extension at 72° C.

All PCR reactions were performed on 5-100 ng in a total volume of 20 μl.

Results

PKR:

Sequencing of the promoter region of the PKR gene in 15 healthy controls revealed a number of sequence variations from the published sequence. In 3′ end of the 5′ untranslated region/exon 1, we found a CGG trincleotide repeat polymorphism. The number of repeats varied between 4 and 10 with 9 repeats being found most commonly, as seen in the published sequence. Each allele was confirmed by cloning and sequencing using the Topo-TA kit (Invitrogen). In addition single nucleotide variants were found at positions −180 (T→G) and −167 (T→C) with respect to the transcription initiation site. At positions −178−[−175] and −169−[−167] we were unable to confirm the published sequence. The nucleotides at position −178−[−175] were consistently found to be CCAA rather than AAAA and at position −169−[−167] was found to be CCT rather than CTG. These appear to be sequencing errors in the published sequence rather than polymorphisms.

No sequence variation was found in the exons encoding the RNA binding region (exons 4-6), the HCV NS5a binding region (exon 11), in the exons encoding the kinase catalytic domain (exons 11, 12, 13) nor in exon 17.

Genotyping of the CGG repeat polymorphism in 5′ untranslated region/exon 1 was performed on the three groups of subjects. Genotyping of a control group was performed to establish the allele frequency in the European population. Allele frequencies were compared between the group with spontaneous elimination of HCV and the group with persistent HCV infection. Allele frequencies are given in Table 2.

Alleles with less than eight CGG repeats were found more frequently in the self-limiting infection group than in the persistent infection group: odds ratio 1.96 (95% confidence interval 1.01-3.85), P=0.02. However, when examining individual alleles only the (CGG)₉ allele was found significantly more frequenly in the persistently infected group (91%) than in the self-limiting infection group (83.7%): χ²=, P=0.02. After applying the Bonferroni correction the result is no longer significant (data not shown).

The GG-genotype of the G(−180)T polymorphism in the PKR promoter was found in 8 out of 45 patients (17.8%) who did not respond to IFN treatment compared to 2 out of 27 patients (7.4%) who had a sustained response to treatment; odds ratio=2.7, 95% confidence interval 0.47-20.2, p-0.22)

OAS-1:

The GG genotype of the A/G polymorphism in the 3′UTR of OAS-1 was found in 11 of 111 patients (9.9%) with self-limiting infection compared to 103 in 453 patients with persistent infection (18.5%); odds ratio 0.48, 95% confidence interval 0.37-0.75, p=0.003).

The other 3 polymorphisms were not associated to outcome of the disease or treatment.

MxA:

The TT genotype in position −88 upstream of the transcription start site in the MxA promoter was found in 6 of 151 patients (4.0%) with self limiting infection compared to 6 of 318 patients (1.9%) with persistent infection odds ratio 2.2; 95% confidence interval 0.6-7.68, p=0.18).

The distribution of genotype and allele frequencies in position −168 was not correlated with the outcome of the disease or treatment.

Discussion

Our data confirm the presence of the CGG trinucleotide repeat polymorphism in the 5′ untranslated region of the PKR gene, as reported by Xu et al, (1998) J. Interferon Cytokine Res.; 18; 609-616. In addition we report two SNP's at position −167 and −180 with respect to the transcription start site. Although the consequences of individual point mutations cannot be predicted a trinucleotide repeat polymorphism in this region may be expected to have functional consequences on the level of gene expression. In other examples of trinucleotide repeats in the 5′ untranslated region or promoter region of a gene increasing numbers of repeats are associated with a diminution or absence of gene expression. The classic examples of this being a CCG repeat in the FMR1 gene in the fragile X syndrome and the CAG repeat in Huntington's Disease (Norremolle et al (1993) Hum. Mol. Genet.; 2; 1475-1476; Kremer et al (1991) Science; 252; 1711-1714).

Our data shows that the shorter length of CGG repeats are associated with self-limiting infection and may be consistent with higher levels of PKR expression overwhelming the inhibition mediated by the HCV NS5a polypeptide. However, the number of trinucleotide repeats normally associated with inhibition of gene transcription is an order of magnitude higher than we have found in these subjects with persistent HCV infection. PKR has been shown to induce apoptosis and inhibition of PKR expression could therefore promote carcinogenesis (Gale et al (1999) J. Virol; 73; 6506-6516). DNA was extracted from 10 HCV related hepatocellular carcinomas and genotyped to determine whether somatic mutations occurred with immense expansion of the CGG repeats. No somatic mutations were identified and the longest series of repeats was only ten.

MxA

The association (SNP G/T-88) in the Caucasian population is stronger with the outcome of the disease (self-limiting versus persistant) than the association between this polymorphism and the outcome of treatment (Response versus Non-Response). However, the association with the outcome of treatment (Response versus Non-response) goes in the same direction as in Japanese population. The observation is due to difference in the allele frequency of the G and T allele in Caucasians, with the T-allele being much rarer in the latter. We therefore sought further polymorphism in the promoter region of Caucasian individuals. Although we found a new SNP at −545, this was not found to be associated with specific outcomes of HCV infection.

OAS-1:

Our data suggest that the 3′UTR SNP in OAS-1 is important in determining the natural outcome of HCV infection. The functional consequences of this polymorphism are unknown but it can be inferred from other studies that changes in the 3′UTR sequences may influence the stability of mRNA and hence the magnitude or duration of protein expression (Goto Y, Yue L, Yokoi A, Nishimura R, Uehara T, Koizumi S, Saikawa Y. (2001) Clin Cancer Research vol 7(7): 1952-6. Alternatively the SNP may be in linkage disequilibrium with a functional polymorphism elsewhere in the gene which we have not yet identified. Interestingly the OAS-1 gene lies on a section of chromosome 12 which is homologeous to the region on mouse chromosome 5 where a flavivirus resistance locus resides (Urosevic N, Silvia O J, Sangster M Y, Mansfield J P, Hodgetts S I, Shellam G R. (1999) J Gen Virol. vol 80 (Pt4): 897-906

TABLE 1

| | | |
|---|---|---|
| Region | Label | Sequence |

| | | |
|---|---|---|
| Promoter region | PPKR3S | AGGGTTCCTGGCCGTGCAGG |
| including exon 1 | | |
| Promoter region | PPKR4A | CCGCGCTCCCTCGGCTGC |
| including exon 1 | | |
| exon 4 | PKR4S | ATATTCTCTTTGTAATCAGG |
| exon 4 | PKR4A | AAAAATGGCAATCACTCACC |
| exon 6 | PKR6S | CCTTCTATGATTTCTCCTAG |
| exon 6 | PKR6A | ATCCAAAGGCAATACGTACC |
| exon 11 | PKR11S | ACAGTGTTTTATCTTTAAGG |
| exon 11 | PKR11A | GTAACATTTACTACTTACTCG |
| exon 12 | PKR12S | CCCTGTTCCTTTTAACTAGG |
| exon 12 | PKR12A | CTCAGGATCATAATCACTGC |
| exon 13 | PKR13S | CTGTGAATTTTATACCCAGG |
| exon 13 | PKR13A | GTATTACTTTTTCCACTTACC |
| exon 17 | PKR17S | GACTCTCACTGTCATTGCAG |
| exon 17 | PKR17A | GTGTCATTGCACTCCAGCCT |

TABLE 2
Allele frequencies for CGG repeats in
the PKR 5′ untranslated region

| | | | |
|---|---|---|---|
| | | Self Limiting | Persistent |
| Number of | Healthy Controls | Infection | Infection |
| Repeats | N (%) | N (%) | N (%) |

| | | | | | | |
|---|---|---|---|---|---|---|
| 4 | 7 | (2.3) | 7 | (3.9) | 3 | (1.3) |
| 5 | 3 | (1) | 2 | (1.1) | 0 | (0) |
| 6 | 18 | (6.8) | 17 | (9.5) | 12 | (5.5) |
| 7 | 0 | (0) | 0 | (0) | 0 | (0) |
| 8 | 1 | (0.4) | 1 | (0.6) | 3 | (1.3) |
| 9 | 231 | (87.5) | 149 | (83.7)* | 197 | (91)* |
| 10 | 4 | (1.5) | 2 | (1.1) | 1 | (0.5) |
| Totals | 264 | | 178 | | 216 | |

*Odds ratio 0.5 (95% confidence interval 0.26-0.96); P = 0.02

TABLE 3
Genotype frequency of T(−180)G polymorphism

| | Self | | | | | | |
|---|---|---|---|---|---|---|---|
| PKR<473> | limiting | Persistent | SR | RR | NR | Fast | Slow |
| genotype | N = 69 (%) | N = 83 | N = 27 | N = 2 | N = 45 | N = 21 | N = 8 |
| GG | 6 | 10 | 2 | 0 | 8 | 3 | 2 |
| | (8.7) | (12.0) | (7.4) | (0.0) | (17.8) | (14.3) | (25.0) |
| GT | 26 | 23 | 9 | 0 | 11 | 6 | 2 |
| | (37.7) | (27.7) | (33.3) | (0.0) | (24.4) | (28.6) | (25.0) |
| TT | 37 | 50 | 16 | 2 | 26 | 12 | 4 |
| | (53.6) | (60.2) | (59.3) | (100) | (57.8) | (57.1) | (50.0) |
| | P = 0.39 | | P = 0.4 (SR vs NR) | | | P = 0.7 | |
| GG | 6 | 10 | 2 | | 8 | 3 | 2 |
| | (8.7) | (12.0) | (7.4) | | (17.8) | (14.3) | (25.0) |
| GT + TT | 63 | 73 | 25 | | 37 | 18 | 6 |
| | (91.3) | (88.0) | (92.6) | | (82.2) | (85.7) | (75.0) |
| | P = 0.5 | | P = 0.22 | | | P = 0.49 | |
| | OR = 0.7 | | OR = 0.37 | | | OR = 0.50 | |
| TT | 37 | 50 | 16 | | 26 | 12 | 4 |
| | (53.6) | (60.2) | (59.3) | | (57.8) | (57.1) | (50.0) |
| GT + GG | 32 | 33 | 11 | | 19 | 9 | 4 |
| | (46.4) | (39.8) | (40.7) | | (42.2) | (42.9) | (50.0) |
| | P = 0.41 | | P = 0.90(SR vsNR) | | | P = 0.73 | |
| | OR = 0.76 | | | | | OR = 1.33 | |
| alleles | | | | | | | |
| G | 38 | 43 | 13 | | 27 | 12 | 6 |
| | (27.5) | (25.9) | (24.1) | | (30.0) | (28.6) | (37.5) |
| T | 100 | 123 | 41 | | 63 | 30 | 10 |
| | (72.2) | (74.1) | (75.9) | | (70.0) | (71.4) | (62.5) |

NR = Non -response (to IFN treatment)
RR = relapsed response
SR = sustained response
slow = slow progression of fibrosis
fast = fast progression of fibrosis.

TABLE 4
Genotype frequency of T(−167)C polymorphism

| | Self | | | | | | |
|---|---|---|---|---|---|---|---|
| PKR<486> | limiting | Persistant | SR | RR | NR | Fast | Slow |
| genotype | N = 87 (%) | N = 217 | N = 82 | N = 35 | N = 88 | N = 43 | N = 23 |
| CC | 11 | 41 | 12 | 7 | 20 | 12 | 3 |
| | (12.6) | (18.9) | (14.6) | (20) | (22.7 | (27.9) | (13.0) |
| CT | 46 | 85 | 34 | 14 | 29 | 13 | 10 |
| | (52.9) | (39.9) | (41.5) | (40.0) | (33.0) | (30.2) | (43.5) |
| TT | 30 | 91 | 36 | 14 | 39 | 18 | 10 |
| | (34.5) | (41.9) | (43.9) | (40.0) | (44.3) | (41.9) | (43.5) |
| | P = 0.08 | | P = 0.31(SR vs NR) | | | P = 0.32 | |
| alleles | | | | | | | |
| C | 68 | 167 | 58 | 28 | 69 | 12 | 6 |
| | (39.1) | (38.5) | (35.4) | | (39.2) | (28.6) | (37.5) |
| T | 106 | 267 | 106 | 42 | 107 | 30 | 10 |
| | (60.9) | (61.5) | (64.6) | | (60.8) | (71.4) | (62.5) |

NR = Non -response (to IFN treatment)
RR = relapsed response
SR = sustained response
slow = slow progression of fibrosis
fast = fast progression of fibrosis.

TABLE 5
Genotype frequency of OAS-1 3′UTR polymorphism

| OAS-1 | Self | | | | | | |
|---|---|---|---|---|---|---|---|
| 3′UTR | limiting | Persistent | SR | RR | NR | Fast | Slow |
| genotype | N = 111 (%) | N = 556 | N = 148 | N = 138 | N = 173 | N = 88 | N = 55 |
| AA | 33 | 202 | 51 | 51 | 64 | 31 | 13 |
| | (29.7) | (36.3) | (34.5) | (43.8) | (37.0) | (35.2) | (23.6) |
| AG | 67 | 251 | 62 | 63 | 84 | 39 | 30 |
| | (60.4) | (45.1) | (41.9) | (31.3) | (48.6) | (44.3) | (54.5) |
| GG | 11 | 103 | 35 | 24 | 25 | 18 | 12 |
| | (9.9) | (18.5) | (23.6) | (25) | (14.5) | (20.5) | (21.8) |
| | P = 0.008 | | P = 0.1037 (SR vs NR) | | | P = 0.32 | |
| | | | P = 0.23 (SR + RRvsNR) | | | | |
| AA | 33 | 202 | 51 | 7 | 64 | 31 | 13 |
| | (29.7) | (36.3) | (34.5) | | (37.0) | (35.2) | (23.6) |
| AG + GG | 78 | 123 | 97 | 9 | 109 | 57 | 42 |
| | (70.3) | (63.7) | (65.5) | | (63.0) | (64.8) | (76.4) |
| | P = 0.18 | | P = 0.64 | | | P = 0.14 | |
| | OR = 0.74 | | OR = 0.90 | | | OR = 1.76 | |
| | | | P = 0.77(R vs NR) | | | | |
| | | | OR = 0.94 | | | | |
| GG | 11 | 103 | 35 | 4 | 25 | 18 | 12 |
| | (9.9) | (18.5) | (23.6) | | (14.5) | (20.5) | (21.8) |
| AA + AG | 100 | 453 | 113 | 12 | 148 | 70 | 43 |
| | (90.1) | (81.5) | (76.4) | | (85.5) | (79.5) | (78.2) |
| | P = 0.003 | | P = 0.047(SR vs NR) | | | P = 0.85 | |
| | OR = 0.48 | | OR = 1.83 | | | OR = 0.92 | |
| alleles | | | | | | | |
| A | 133 | 655 | 58 | 28 | 69 | 101 | 56 |
| | (59.9) | (58.9) | (35.4) | | (39.2) | (57.4) | (50.9) |
| | 89 | 457 | 106 | 42 | 107 | 75 | 54 |
| G | (40.1.9 | (41.1) | (64.6) | | (60.8) | (42.6) | (49.1) |

| | | |
|---|---|---|
| P = 0.78 | P = 0.13 (SR vsNR) | P = 0.2842 |
| OR = 1.04 | OR = 0.72 | OR = 1.3 |

NR = Non -response (to IFN treatment)
RR = relapsed response
SR = sustained response
slow = slow progression of fibrosis
fast = fast progression of fibrosis.

TABLE 6
Genotype frequency of MxA(−88) polymorphism

| | Self | | | | | | |
|---|---|---|---|---|---|---|---|
| PKR<486> | limiting | Persistant | SR | RR | NR | Fast | Slow |
| genotype | N = 151 (%) | N = 318 | N = 98 | N = 58 | N = 135 | N = 57 | N = 46 |
| GG | 100 | 21 | 72 | 42 | 109 | 44 | 33 |
| | (66.2) | (75.8) | (73.5) | (72.4) | (80.7) | (77.2) | (71.7 |
| GT | 45 | 71 | 2 | 15 | 23 | 12 | 12 |
| | (29.8) | (22.3) | (24.5) | (25.9) | (17.0) | (21.1) | (26.1) |
| TT | 6 | 6 | 2 | 1 | 3 | 1 | 1 |
| | (4.0) | (1.9) | (2.0) | (1.7) | (2.2) | (1.8) | (2.2) |
| | P = 0.069 | | P = 0.31(SR vs NR) | | | P = 0.32 | |
| GG | 100 | 241 | 72 | | 109 | 44 | 33 |
| | (66.2) | (75.8) | (73.5) | | (80.7) | | |
| GT + TT | 51 | 77 | 26 | | 26 | 13 | 13 |
| | (33.8) | (24.2) | (26.5) | | (19.3) | | |
| | P = 0.03 | | P = 0.19 | | | P = 0.53 OR = 1.33 | |
| | OR = 0.63 | | | | | | |
| TT | 6 | 6 | 2 | | 3 | 1 | 1 |
| | (4.0) | (1.9) | (2.0) | | (2.2) | | |
| GT + GG | 145 | 312 | 96 | | 132 | 56 | 46 |
| | (96.0) | (98.1) | (98) | | (97.8) | | |
| | P = 0.18 | | | | | P = 0.88 | |
| | OR = 2.15 | | | | | OR = 0.8 | |
| alleles | | | | | | | |
| G | 245 | 553 | 168 | 28 | 241 | 100 | 78 |
| | (81.1) | (86.9) | (85.7) | | (89.3) | (87.7) | (84.8) |
| T | 57 | 83 | 28 | 42 | 29 | 14 | 14 |
| | (18.9) | (13.1) | (14.3) | | (10.7) | (12.3) | (15.2) |

NR = Non -response (to IFN treatment)
RR = relapsed response
SR = sustained response
slow = slow progression of fibrosis
fast = fast progression of fibrosis.

TABLE 7
Genotype frequency of MxA(−545) polymorphism

| | Self | | | | | | |
|---|---|---|---|---|---|---|---|
| PKR<486> | limiting | Persistent | SR | RR | NR | Fast | Slow |
| genotype | N = 95 (%) | N = 159 | N = 74 | N = 3 | N = 79 | N = 38 | N = 22 |
| AA | 16 | 42 | 22 | 1 | 17 | 8 | 5 |
| | (16.8) | (26.4) | (29.7) | (33.3) | (21.5) | (21.1) | (22.7) |
| AG | 60 | 93 | 41 | 1 | 50 | 26 | 16 |
| | (63.2) | (58.5) | (55.4) | (33.3) | (63.3) | (68.4) | (72.7) |
| GG | 19 | 24 | 11 | 1 | 12 | 4 | 1 |
| | (20.0) | (15.1) | (14.9) | (33.3) | (15.2) | (10.5) | (4.5) |
| | P = 0.1784 | | P = 0.49(SR vs NR) | | | P = 0.72 | |
| AA | 16 | 42 | 22 | | 17 | 8 | 5 |
| | (16.8) | (26.4) | (29.7) | | (21.5) | (21.1) | (22.7) |
| AG + GG | 79 | 117 | 52 | | 62 | 30 | 17 |
| | (83.2) | (73.6) | (70.3) | | (78.5) | (78.9) | (77.3) |
| | P = 0.08; OR = 0.56 | | P = 0.24 | | | P = 0.88 | |
| | | | OR = 1.54 | | | OR = 0.91 | |
| GG | 19 | 24 | 11 | | 12 | 4 | 1 |
| | (20.0) | (15.1) | (14.9) | | (15.2) | (10.5) | (4.5) |
| AG + AA | 76 | 135 | 63 | | 67 | 34 | 21 |
| | (80.0) | (84.9) | (85.1) | | (84.8) | (89.5) | (95.5) |
| | P = 0.31; OR = 1.41 | | P = 0.96; OR = 0.97 | | | P = 0.42 | |
| | | | | | | OR = 2.47 | |
| alleles | | | | | | | |
| A | 92 | 177 | 85 | 28 | 84 | 42 | 26 |
| | (48.4) | (55.7) | (57.4) | | (53.2) | (55.3) | (59.1) |
| G | 98 | 141 | 106 | 63 | 74 | 34 | 18 |
| | (51.6)) | (44.3) | (42.6) | | (46.8) | (44.7) | (40.9) |

NR = Non -response (to IFN treatment)
RR = relapsed response
SR = sustained response
slow = slow progression of fibrosis
fast = fast progression of fibrosis.

### EXAMPLE 2

### Screen for Modifiers of OAS1 Activity In Vitro

The effect of test compounds on the activity of human OAS1 may be measured by an electrophoretic mobility shift assay. Recombinantly expressed and purified OAS1 is exposed to a 100 nM concentration of 110 bp long dsRNA molecules. The dsRNA molecules are radiolabelled using polynucleotide kinase. At this subsaturating concentration the dsRNA molecules are able to active OAS1. This activation is measured by, for example, performing an electrophoretic mobility shift assay on the reaction mixture and measuring the level of OAS1 activity by scintillation counting of the regions of the gel where the OAS1/dsRNA complex resides. Then, using the same quantity of OAS1 protein and dsRNA molecules as described in the above assay, the active OAS1/dsRNA complex is exposed to the test compound, either at a single concentration (for example 30 μM in an initial compound library screen), or at more than one concentration (for example two or more of 3 μM, 10 μM, 30 μM and 100 μM). The tests are generally performed at least in duplicate.

The effect of the test compound on the activity of OAS1 is measured by, for example, performing an electrophoretic mobility shift assay on the reaction mixture and measuring the level of OAS1 activity by scintillation counting of the regions of the gel where the OAS1/dsRNA complex resides.

More details on the preparation of recombinant OAS1 and the assay methods used above can be found in Sarkar and Sen (1998), Methods; 15; 233-242.

### EXAMPLE 3

### Screen for Modifiers of RNAse L Activity In Vitro

The effect of test compounds on the activity of human RNAse L may be measured by an electrophoretic mobility shift assay. Recombinantly expressed and purified RNAse L is exposed to a radiolabelled substrate, for example oligouridylic acid, in the presence of an activating cofactor, for example 0.1 nM of oligomers of 2′,5′-oligosadenylate. At this concentration RNAse L will degrade the substrate which can be measured, for example, by an electrophoretic mobility shift assay on the reaction mixture. In this case the sunstrate will be degraded and the radiolabelled product will migrate towards the bottom of the gel. The activity of RNAse L can be measured by comparing the substrate degradation of reaction mixture with the active cofactor to the level of substrate degradation without the cofactor using, for example, the electrophoretic mobility shift assay described above.

Then, using the same quantity of RNAse L protein and the cofactor molecules as described in the above assay, the active RNAse L protein/cofactor complex is exposed to the test compound, either at a single concentration (for example 30 μM in an initial compound library screen), or at more than one concentration (for example two or more of 3 μM, 10 μM, 30 μM and 100 μM). The tests are generally performed at least in duplicate.

The effect of the test compound on the activity of RNAse L can be measured by comparing the substrate degradation of reaction mixture with the test compound to the level of substrate degradation without the test compound using, for example, the electrophoretic mobility shift assay described above.

Further details on the preparation of recombinant RNAse L, oligomers of 2′,5′-oligosadenylate and the assay methods used above can be found in Rusch et al (2001) Methods Enzymol.; 342;10-20.

### EXAMPLE 4

### A Method to Identify Compounds that Modify the Activity of OAS1 In Vivo

The effect of test compounds on the activity of human OAS1 may be measured in vivo using a human cell line. Cells are incubated in petri dishes in the presence or absence of the test compound, either at a single concentration (for example 30 μM in an initial compound library screen), or at more than one concentration (for example two or more of 3 μM, 101M, 30 μM and 100 μM). The tests are generally performed at least in duplicate.

After 16 hours exposure, the cells are fragmented and the mRNA and protein components purified using methods well known to those skilled in the art.

Using northern blotting or RT-PCR, methods well known to those skilled in the art, it is possible to compare the quantity of OAS1 mRNA transcribed in cell lines with and without exposure to the test compound, and hence measure the effect of the test compound on the activity of the OAS1 gene.

Using western blotting, a method well known to those skilled in the art, it is possible to compare the quantity of OAS1 protein in cell lines with and without exposure to the test compound, and hence measure the effect of the test compound on the activity of the OAS1 gene.

It is also possible to measure the activity of the OAS1 protein present in the cell lines. The OAS1 protein is purified from the cell lines using a immunoprecipitation, a method well known to those skilled in the art. The protein may then be used in an electrophoretic mobility shift assay as described in Example 2.

### EXAMPLE 5

### A Method to Identify Compounds that Modify the Activity of RNAse L In Vivo

The effect of test compounds on the activity of human RNAse L may be measured in vivo using a human cell line. Cells are incubated in petri dishes in the presence of an activating cofactor, for example 0.1 nM of oligomers of 2′, 5′-oligosadenylate, and in the presence or absence of the test compound, either at a single concentration (for example 30 μM in an initial compound library screen), or at more than one concentration (for example two or more of 3 μM, 10 μM, 30 μM and 100 μM). The tests are generally performed at least in duplicate.

After 16 hours exposure, the cells are fragmented and the mRNA and protein components purified using methods well known to those skilled in the art.

Using northern blotting or RT-PCR, methods well known to those skilled in the art, it is possible to compare the quantity of RNAse L mRNA transcribed in cell lines with and without exposure to the test compound, and hence measure the effect of the test compound on the activity of the RNAse L gene.

Using western blotting, a method well known to those skilled in the art, it is possible to compare the quantity of RNAse L protein in cell lines with and without exposure to the test compound, and hence measure the effect of the test compound on the activity of the RNAse L gene.

It is also possible to measure the activity of the RNAse L protein present in the cell lines. Once activated, RNAse L digests all cellular RNA, including rRNA. Hence total RNA can be extracted from the cells and the degree of rRNA degradation measured using northern blotting, using methods well known to those skilled in the art. By comparing the quantity of rRNA degraded in a control sample (with cofactor) to that degraded with a test compound, it is possible to determine whether there is any effect of the test compound on the activity of RNAse L.

Further details on the measurement of RNAse L activity using an rRNA degradation assay can be found in Rusch et al (2001) Methods Enzymol.; 342; 10-20.

### EXAMPLE 6

### A Method to Treat a Patient at or at Risk of HCV Infection

A patient may be diagnosed as having HCV infection using well known methods, including HCV antibody detection and RNA detection. Alternatively or in addition the patient may be at risk of infection by HCV, for example a healthcare worker. The patient is treated with interferon and with a compound which modulates OAS1 and/or RNAse L activity. The patient may be tested in order to determine the OAS1 genotype.

### EXAMPLE 7

### Gene Therapy as a Method Treat a Patient at or at Risk of HCV Infection

A patient may be diagnosed as having HCV infection using well known methods, including HCV antibody detection and RNA detection. Alternatively or in addition the patient may be at risk of infection by HCV, for example a healthcare worker. Should the patient have the ‘G’ genotype of the OAS1 gene, using the method of Example 1, then the patient can be treated to provide the therapeutic ‘A’ genotype of the OAS1 gene. The therapeutic OAS1 gene may be supplied to the patient using the gene therapy methods discussed herein and well known to those skilled in the art.

1-36. (canceled)

37. A method for screening for compounds for treating hepatitis C virus (HCV) infection comprising steps of:
(a) assessing the effect of a test compound on the activity of 2′-5′-oligoadenylate synthetase (OAS), wherein said test compound is one other than a compound selected from the group consisting of interferons and isoprenoids including geranylgeranylacetone (GGA);

38. A method as defined in claim 37 including the step of:
(b) treating a cell with said test compound; and
(c) assessing any change in OAS gene activity and/or OAS protein activity or level.

39. A method as defined in claim 38 wherein the cell is an animal cell.

40. A method as defined in claim 38 wherein the cell is a human cell.

41. A method as defined in claim 38 including the step of using a compound capable of modulating the level of activity of the OAS gene and/or activity of the OAS protein identified in claim 38 in the manufacture of a medicament for the treatment of a patient with or at risk of HCV infection.

42. A compound capable of modulating the level of activity of an entity selected from the group consisting of the OAS gene, the OAS protein and combinations thereof identified or identifiable from the method of claim 37.

43. A method of screening for compounds for treating HCV infection including the step of:
(a) assessing the effect of a test compound on the activity of RNAse L wherein said test compound is one other than a compound selected from the group consisting of interferons and isoprenoids, including geranylgeranylacetone (GGA).

44. A method as defined in claim 43 including the steps of:
(b) treating a cell with a test compound; and
(c) assessing any change in RNAse L gene activity and/or RNAse L protein activity or level.

45. A method as defined in claim 44 wherein the cell is an animal cell.

46. A method as defined in claim 44 wherein the cell is a human cell.

47. A method as defined in claim 44 including the step of using a compound identified in claim 44 in the manufacture of a medicament for the treatment of a patient with or at risk of HCV infection.

48. A compound capable of modulating the level of activity of an entity selected from the group consisting of the RNAse L gene, the RNAse L protein and combinations thereof as identified or identifiable from the method of claim 43.

49. A method of screening for compounds for treating HCV infection including the step of:
(a) assessing the effect of a test compound on the activity of 2′-5′ phosphodiesterase wherein said test compound is one other than a compound selected from the group consisting of interferons and isoprenoids, including geranylgeranylacetone (GGA).

50. A method as defined in claim 49 including the steps of:
(b) treating a cell with a test compound; and
(c) assessing any change in the 2′-5′ phosphodiesterase gene activity and/or 2′-5′ phosphodiesterase protein activity or level.

51. A method as defined in claim 50 wherein the cell is an animal cell.

52. A method as defined in claim 50 wherein the cell is a human cell.

53. A method as defined in claim 50 including the further step of using a compound identified in claim 50 in the manufacture of a medicament for the treatment of a patient with or at risk of HCV infection.

54. A compound capable of modulating the level of activity of an entity selected from the group consisting of the 2′-5′ phosphodiesterase gene, activity of the 2′-5′ phosphodiesterase protein, or a combination thereof as identified or identifiable from the method of claim 49.

55. A method for the manufacture of a product selected from the group consisting of a medicament for the treatment of a patient with or at risk of HCV infection and a diagnostic reagent for use in the assessment or diagnosis of a patient with or at risk of HCV infection including a step using a nucleic acid which hybridizes selectively to an OAS nucleic acid.

56. A method as defined in claim 55 wherein the nucleic acid comprises the polynucleotide sequence shown in FIG. 1 or a fraction thereof wherein the nucleotide sequence at position 84 bp into the untranslated 3′ end of exon 8 is A.

57. A method as defined in claim 55 wherein the nucleic acid comprises the polynucleotide sequence shown in FIG. 1 or a fraction thereof wherein the nucleotide sequence at position 84 bp into the untranslated 3′ end of exon 8 is G.

58. A method of determining whether a patient with or at risk of HCV infection has an OAS1 gene in which the nucleotide sequence at position 84 bp into the untranslated 3′ end of exon 8 is G, wherein the method comprises a step of determining the OAS1 genotype of said patient.

59. A method as defined in claim 58 comprising a step of performing an allele specific PCR reaction using polynucleotides comprising the DNA sequences:
(1) CTCACTGAGGAGCTTTGTCT
(2) CACTGAGGAGCTTTGTCC
and/or
(3) CAGGTGGGACTCTTGATCCAG.

60. A method as defined in claim 58 comprising the further step of determining the relative prospects of recovery from infection and/or success of treatment with interferon of a patient with or at risk of HCV infection based on said determination of the OAS genotype of the patient.

61. A method as defined in claim 59 comprising the further step of determining the relative prospects of recovery from infection and/or success of treatment with interferon of a patient with or at risk of HCV infection based on said determination of the OAS genotype of the patient.

62. A method as defined in claim 60 including the step of selecting a method of treatment of a patient with or at risk of HCV infection.

63. A method as defined in claim 61 including the step of selecting a method of treatment of a patient with or at risk of HCV infection.

64. A pharmaceutical composition comprising:
(a) a compound, polynucleotide or polypeptide comprising a compound selected from the group consisting of compounds that are capable of modulating the level of activity of the OAS1 gene and/or activity of the OAS1 protein, compounds that are capable of modulating the level of activity of the RNAse L gene and/or activity of the RNAse L protein, compounds that are capable of modulating the level of activity of the 2′-5′ phosphodiesterase gene, and/or activity of the 2′-5′ phosphodiesterase protein and/or a recombinant polynucleotide which hybridizes selectively to an OAS nucleic acid;
(b) a therapeutically appropriate quantity of an interferon; and
(c) a pharmaceutically acceptable diluent or carrier.


SEQUENCE LISTING (30 sequences)

SEQ ID NO: 1 - protein, 400 aa, Homo sapiens
MMDLRNTPAKSLDKFIEDYLLPDTCFRMQIDHAIDIICGFLKERCFRGSSYPVCVSKVVKGGSSGKGTTLRGRSDADLVVFLSPLTTFQDQLNRRGEFIQEIRRQLEACQRERALSVKFEVQAPRWGNPRALSFVLSSLQLGEGVEFDVLPAFDALGQLTGSYKPNPQIYVKLIEECTDLQKEGEFSTCFTELQRDFLKQRPTKLKSLIRLVKHWYQNCKKKLGKLPPQYALELLTVYAWERGSMKTHFNTAQGFRTVLELVINYQQLCIYWTKYYDFKNPIIEKYLRRQLTKPRPVILDPADPTGNLGGGDPKGWRQLAQEAEAWLNYPCFKNWDGSPVSSWILLAESNSTDDETDDPRTYQKYGYIGTHEYPHFSHRPSTLQAASTPQAEEDWTCTIL

SEQ ID NO: 2 - DNA, 1590 bp, Homo sapiens
gaggcagttctgttgccactctctctcctgtcaatgatggatctcagaaataccccagccaaatctctggacaagttcattgaagactatctcttgccagacacgtgtttccgcatgcaaatcgaccatgccattgacatcatctgtgggttcctgaaggaaaggtgcttccgaggtagctcctaccctgtgtgtgtgtccaaggtggtaaagggtggctcctcaggcaagggcaccaccctcagaggccgatctgacgctgacctggttgtcttcctcagtcctctcaccacttttcaggatcagttaaatcgccggggagagttcatccaggaaattaggagacagctggaagcctgtcaaagagagagagcactttccgtgaagtttgaggtccaggctccacgctggggcaacccccgtgcgctcagcttcgtactgagttcgctccagctcggggagggggtggagttcgatgtgctgcctgcctttgatgccctgggtcagttgactggcagctataaacctaacccccaaatctatgtcaagctcatcgaggagtgcaccgacctgcagaaagagggcgagttctccacctgcttcacagaactacagagagacttcctgaagcagcgccccaccaagctcaagagcctcatccgcctagtcaagcactggtaccaaaattgtaagaagaagcttgggaagctgccacctcagtatgccctggagctcctgacggtctatgcttgggagcgagggagcatgaaaacacatttcaacacagcccaaggatttcggacggtcttggaattagtcataaactaccagcaactctgcatctactggacaaagtattatgactttaaaaaccccattattgaaaagtacctgagaaggcagctcacgaaacccaggcctgtgatcctggacccggcggaccctacaggaaacttgggtggtggagacccaaagggttggaggcagctggcacaagaggctgaggcctggctgaattacccatgctttaagaattgggatgggtccccagtgagctcctggattctgctggctgaaagcaacagtacagacgatgagaccgacgatcccaggacgtatcagaaatatggttacattggaacacatgagtaccctcatttctctcatagacccagcacgctccaggcagcatccaccccacaggcagaagaggactggacctgcaccatcctctgaatgccagtgcatcttgggggaaagggctccagtgttatctggaccagttccttcattttcaggtgggactcttgatccagagaagacaaagctcctcagtgagctggtgtataatccaagacagaacccaagtctcctgactcctggccttctatgccctctatcctatcatagataacattctccacagcctcacttcattccacctattctctgaaaatattccctgagagagaacagagagatttagataagagaatgaaattccagccttgactttcttctgtgcacctgatgggagggtaatgtctaatgtattatcaataacaataaaaataaagcaaataccaaaaa

SEQ ID NO: 3 - DNA, 20 bp, Artificial
  PCR primer based on Homo sapiens OAS1.
ctcactgaggagctttgtct

SEQ ID NO: 4 - DNA, 18 bp, Artificial
  PCR primer based on Homo sapiens OAS1.
cactgaggagctttgtcc

SEQ ID NO: 5 - DNA, 21 bp, Artificial
  PCR primer based on Homo sapiens OAS1.
caggtgggactcttgatccag

SEQ ID NO: 6 - DNA, 20 bp, Artificial
  PCR primer based on Homo sapiens PKR.
agggttcctggccgtgcagg

SEQ ID NO: 7 - DNA, 18 bp, Artificial
  PCR primer based on Homo sapiens PKR.
ccgcgctccctcggctgc

SEQ ID NO: 8 - DNA, 20 bp, Artificial
  PCR primer based on Homo sapiens PKR.
atattctctttgtaatcagg

SEQ ID NO: 9 - DNA, 20 bp, Artificial
  PCR primer based on Homo sapiens PKR.
aaaaatggcaatcactcacc

SEQ ID NO: 10 - DNA, 20 bp, Artificial
  PCR primer based on Homo sapiens PKR.
ccttctatgatttctcctag

SEQ ID NO: 11 - DNA, 20 bp, Artificial
  PCR primer based on Homo sapiens PKR.
atccaaaggcaatacgtacc

SEQ ID NO: 12 - DNA, 20 bp, Artificial
  PCR primer based on Homo sapiens PKR.
acagtgttttatctttaagg

SEQ ID NO: 13 - DNA, 21 bp, Artificial
  PCR primer based on Homo sapiens PKR.
gtaacatttactacttactcg

SEQ ID NO: 14 - DNA, 20 bp, Artificial
  PCR primer based on Homo sapiens PKR.
ccctgttccttttaactagg

SEQ ID NO: 15 - DNA, 20 bp, Artificial
  PCR primer based on Homo sapiens PKR.
ctcaggatcataatcactgc

SEQ ID NO: 16 - DNA, 20 bp, Artificial
  PCR primer based on Homo sapiens PKR.
ctgtgaattttatacccagg

SEQ ID NO: 17 - DNA, 21 bp, Artificial
  PCR primer based on Homo sapiens PKR.
gtattactttttccacttacc

SEQ ID NO: 18 - DNA, 20 bp, Artificial
  PCR primer based on Homo sapiens PKR.
gactctcactgtcattgcag

SEQ ID NO: 19 - DNA, 20 bp, Artificial
  PCR primer based on Homo sapiens PKR.
gtgtcattgcactccagcct

SEQ ID NO: 20 - DNA, 624 bp, Homo sapiens
ggatccagatggcatgtcacagtatactaaatgctcactgaatccagctgcaatgcaggaagactcccctgatgtgatcatgtgtctcaccctttcaggctgaaagcaacagtacagacgatgagaccgacgatcccaggacgtatcagaaatatggttacattggaacacatgagtaccctcatttctctcatagacccagcacgctccaggcagcatccaccccacaggcagaagaggactggacctgcaccatcctctgaatgccagtgcatcttgggggaaagggctccagtgttatctggaccagttccttcattttcaggtgggactcttgatccagagaagacaaagctcctcagtgagctggtgtataatccaagacagaacccaagtctcctgactcctggccttctatgccctctatcctatcatagataacattctccacagcctcacttcattccacctattctctgaaaatattccctgagagagaacagagagatttagataagagaatgaaattccagccttgactttcttctgtgcacctgatgggagggtaatgtctaatgtattatcaataacaataaaaataaagcaaataccatttattgggtg

SEQ ID NO: 21 - DNA, 19 bp, Artificial
  PCR primer based on Homo sapiens MxA.
ggcctggcctgacaactat

SEQ ID NO: 22 - DNA, 19 bp, Artificial
  PCR primer based on Homo sapiens MxA.
catccaagcctgcacgtat

SEQ ID NO: 23 - DNA, 20 bp, Artificial
  PCR primer based on Homo sapiens MxA.
gctttgtgtgagcaacatgg

SEQ ID NO: 24 - DNA, 20 bp, Artificial
  PCR primer based on Homo sapiens MxA.
ggctcatctggtctctccag

SEQ ID NO: 25 - DNA, 19 bp, Artificial
  PCR primer based on Homo sapiens PKR.
gactaggccagcggagaac

SEQ ID NO: 26 - DNA, 18 bp, Artificial
  PCR primer based on Homo sapiens PKR.
gcttcgggagagctggtt

SEQ ID NO: 27 - DNA, 20 bp, Artificial
  PCR primer based on Homo sapiens MxA.
tgaagacccccaattaccaa

SEQ ID NO: 28 - DNA, 20 bp, Artificial
  PCR primer based on Homo sapiens MxA.
ctctcgttcgcctctttcac

SEQ ID NO: 29 - DNA, 19 bp, Artificial
  PCR primer based on Homo sapiens MxA.
ggcctggcctgacaactat

SEQ ID NO: 30 - DNA, 19 bp, Artificial
  PCR primer based on Homo sapiens MxA.
catccaagcctgcacgtat